(19) United States
(12) Patent Application Publication
(10) Pub. No.: US 2024/0281561 A1
(43) Pub. Date: Aug. 22, 2024

(54) MEDICAL DATA SHARING USING BLOCKCHAIN

(71) Applicants: Carl Zeiss Meditec, Inc., Dublin, CA (US); Carl Zeiss Meditec AG, Jena (DE)

(72) Inventors: Hugang Ren, Dublin, CA (US); Neil DSouza, Dublin, CA (US); Jeffrey Schmidt, San Ramon, CA (US)

(73) Assignees: Carl Zeiss Meditec, Inc., Dublin, CA (US); Carl Zeiss Meditec AG, Jena, TH (DE)

(21) Appl. No.: 18/569,995

(22) PCT Filed: Jun. 16, 2022
(86) PCT No.: PCT/EP2022/066472

§ 371 Date: Dec. 13, 2023

Related U.S. Application Data
(60) Provisional application No. 63/211,955, filed on Jun. 17, 2021.

(51) Int. Cl. G06F 21/62; G06F 21/60; G16H 10/60

(52) U.S. Cl. CPC G06F 21/6245; G06F 21/602; G16H 10/60

(57) ABSTRACT

A system/method for transacting medical data among different stakeholders (remote users or subscribers) uses a blockchain computer network to record all data transactions and ensure privacy of the data owners.

### FIELD OF INVENTION

The present invention is generally directed to the use of blockchain for medical applications.

### BACKGROUND

Data plays an important role in developing artificial intelligence solutions and analytics, as well as building smart devices and services. Sharing data among doctors, researchers and commercial organizations can advance new discoveries and provide better diagnosis and treatment in medicine. Specifically, sharing data among ophthalmic community has the potential to enable better eye care for patients.

There are two primary issues with using patient data to improve clinical outcomes: 1) technical access to the data across a range of storage locations, applications and IT/security frameworks and 2) increase in global patient (consumer) privacy protection through an ever changing, more impactful, highly disparate regulatory landscape.

There are obstacles to affecting access to patient medical data, such as ophthalmic data, orthopedic data, cardiology data, dermatology data, radiology, etc. For example, patient medical data is usually stored at each hospital, imaging center or doctor's office where the data was originally acquired (e.g., the medical institution that collected the medical data) or more recently in an electronic medical record system possibly at a virtual machine (VM) hosting site (cloud). Due to the technical complications in storage infrastructure, software/databases, local security infrastructure (e.g. firewalls, authentications, encryption, etc.), legal privacy concerns, data size, control issues sharing data with other doctors, trust, and increasing patient concern over their digital footprints, the cost to enable data access has generally outweighed the value to make the data accessible.

Other issues are raised by the regulatory landscape. Today, accessible biometric data cannot be ‘legally used’ if the patient's consent is missing. Data collected over a period when global patient/consumer privacy frameworks have been developing and practice/doctor consent rigor has resulted in challenges to using the existing data collected and created challenges going forward with new data. Even in cases where informed patient consent does exist, doctors, hospitals, and other medical institutions are still not motivated to share the medical data since the incentives are case-by-case and difficult to negotiate at large scale. Additionally, for ‘consumers of the data’ managing both the data set and the legal consent documentation poses significant administrative/legal challenges. For example, there are questions, such as, “Is the consent generated by another institute at a specific time period legally viable today?”

Approaches to address one or more of the above issues have been previously proposed. Some examples include: “A Framework for Secure and Decentralized Sharing of Medical Imaging Data Via Blockchain Consensus”, Health Informatics J., 2019 Dec, 25(4): 1398-1411, doi: 10.1177/1460458218769699; A. Azaria, A. Ekblaw, T. Vieira and A. Lippman, “MedRec: Using Blockchain for Medical Data Access and Permission Management,” 2016 2 nd International Conference on Open and Big Data (OBD), Vienna, 2016, pp. 25-30, doi: 10.1109/OBD.2016.11; and Fan, K., Wang, S., Ren, Y. et al., “MedBlock: Efficient and Secure Medical Data Sharing Via Blockchain,” J Med Syst, 42, 136 (2018), doi:/10.1007/s10916-018-0993-7.

It is an object of the present invention to provide a system and method for providing valid, real-time, patient/consumer consent for accessing patient medical data/records.

It is another object of the present invention to provide a system and method to ensure real-time compliance with legal/regulatory requirements.

It is a further object of the present invention to provide a method and system to facilitate and incentivize patients/consumers and (e.g., medical) institutions to share their data, and to gain access to archival (e.g., historically collected) data.

### SUMMARY OF INVENTION

As the volatility of the global regulatory framework is not expected to stabilize anytime soon, a solution that can enable real-time, patient/consumer confirmed consent is needed. In addition to ensuring compliance to real-time legal requirements, a solution that would incentivize patients/consumers to share their data, opens up access to historically collected data, and incentivizes institutions to share their data is needed. The above objects are met in a method/system based on the use of blockchain technology to provide a framework to achieve this solution.

In the present invention, a marketplace for data sharing is established to allow a patient to commercialize his/her own medical data/records with market value within the medical community using blockchain technology. As an example, the present invention is described as applied to the ophthalmic medical community, but it is to be understood that the present invention may be applied to other medical fields, or other more general data collection fields. The present invention also provides a platform for researchers and organizations to purchase needed data. At the same time, all stakeholders, such as the patient, the doctor, the data host, the medical institution, and the blockchain service provider (e.g., a managing entity or workplace platform or marketplace platform or blockchain administrator or data broker) get a portion of the payment as incentive.

With the transaction happening in the marketplace, multiple different transaction types (both monetary and nonmonetary) can be defined to limit the consent (by the owner of the medical data) as well as to provide different price tiers for the need (e.g., different data-access privileges). Such transaction types can include view only, rent/lease with expiration, permanent download, processing in the (workplace/marketplace) platform (e.g., the managing entity controls/manages the processing of select medical data remote from the entity that requests access to the medical data), etc.

To make sure the data shared is consistent with the original data, a data validation mechanism is built into the network to verify that the data that is stored off the blockchain is always matching the meta data on the blockchain. For example, the meta data on the blockchain may include an electronic ledger (e.g., description/summary of the stored medical data/records), while the medical data itself is stored by a separate data host (e.g., under control of the managing entity), and a hashing algorithm (or other verification method) may be used to confirm that the meta data on the blockchain matches the separately stored medical data. In a similar manner, the present managing entity may use a validation mechanism, such as a hashing algorithm, to check that the data that a member user (e.g., remote user) gains access to is the same as when the data was originally stored by the managing entity.

The blockchain (computer) network can be integrated into a medical data acquisition device, such as an ophthalmic imaging devices, directly, and the acquired medical data (i.e. measurements, images, etc.) may be automatically sent/transmitted by the medical device to the managing entity with consent from the patient (either prior to, or after, acquisition of the medical data). Therefore, medical data is seamlessly saved/recorded to the blockchain (and/or managing entity) without affecting any current clinical workflow.

Based on this framework, referrals between doctors with data sharing between doctors can also be implemented via the present blockchain construct. Since the patient is at the center of the blockchain, the patient can therefore access their own medical data at any time. As all members have access to the blockchain ledger, transparency and trust is built into the present network between blockchain members.

This novel approach provides a way to share imaging data (e.g., within the ophthalmic or other medical community) using a marketplace that provides financial incentives for all stakeholders. The present invention also provides different data-access types/formats/methods, including data validation. The present novel approach also enables ophthalmic patients to fully access their imaging data (or other medical data) and have full control over their data access.

Thus, the present invention provides a method and system for implementing a data transaction (e.g., a transaction of medical data), wherein with agreement of an owner of the medical data (e.g., the patient that is the subject of, or described by, the medical data), a managing entity (workplace platform/marketplace platform/blockchain service provider/blockchain administrator) receives the medical data for storage. The medical data may be received electronically (e.g., via a computer network, such as the internet using an encryption technology) or on an electronic data storage medium (e.g., CD/DVD). The managing entity records a summary of the received medical data to a blockchain that maintains at least an electronic ledger of available medical records (e.g., the medicals records that are accessible via the managing entity). A remote user can then transmit a request for medical data to the managing entity, and preferably specify criteria describing the type of medical data that is being requested. The managing entity may respond to the electronic request from the remote user for medical data meeting user-specified criteria, by providing the remote user with a listing (e.g., a count) of available qualifying medical records that meet the user-specified criteria, as determined at least in part from the electronic ledger. The remote user may select from the listing or simply specify a number of desired medical records. The managing entity may respond to the remote user selecting one or more of the qualifying medical records by granting the remote user access to the selected qualifying medical records in accordance with an access-approval status for each qualifying medical record granted by the owner of the qualifying medical record and recording the data transaction to the blockchain. The access-approval status may have been provided by the data owner prior to the remote user requesting the data. For example, the data owner may be provided pre-approval for select doctors/institutes, or for doctor referrals, or for specific use of their medical data, etc.

Optionally, the managing entity may provide different types of access permissions to the stored medical data. In this case, the managing entity may receive from the remote user an access-type request indicating the type of data access being requested. The access-type request may include one or more of data viewing (only), temporary data access with expiration period, permanent data access with data download, and data processing remote from the remote user and managed by the managing entity (e.g., a data processing service provided by the managing entity). The option for permanent access with data download may include removal of the accessed qualifying medical record from the available medical records in the electronic ledger (e.g., the downloaded data may no longer available on the blockchain). If desired, each access-type may have an associated access-price payable by the remote user.

In some embodiments, the access option for data processing managed by the managing entity may include generation of a machine learning model using the selected medical records and granting the remote user access to the generated machine learning model. For example, the machine model options may include a deep learning model selected from one or more of an artificial neural network, convolutional neural network, u-net, recurrent neural networks, generative adversarial networks, and multilayer perceptron's. Alternatively, or additionally, the machine model option may include a machine learning model based one or more of a classification model, regression model, clustering, and dimensionality reduction.

As discussed above, the medical data may include medical measurements or images taken by a medical device (e.g., radiology equipment, computed tomography device, optical coherence tomography device, fundus imager, visual field test instrument (perimeter) for testing a patient's visual field, or a slit scanning ophthalmic system), and the medical device automatically sends the medical data to the managing entity with consent from the patient.

The owner of the medical data may be identified by a public identifier based on a public key that is associated with a corresponding private key, where the public identifier excludes personal identifying information. In this manner, the managing entity maintains hidden any personal identifying information of the owner of the medical data. In other words, no remote user can personally identify any owner of stored medical data.

Optionally, the listing of available qualifying medical records that meet the user-specified criteria may include a count of qualifying medical records. If the number is not sufficient for the remote user, the remote user may choose to alter the user-specified criteria, such as by reducing the number of user-specified criteria. The managing entity may then respond to an electronic altering of the user-specified criteria by providing the remote user with an updated listing of available qualifying medical records that meet the altered user-specified criteria, which may be higher than previously presented.

The remote user may select individual medical records, but may also select a number, or provide a number, of desired medical records that may constitute a lot, or group. In response to the remote user selecting one or more of the qualifying medical records, the managing entity may check for a pre-existing access-approval status for each of the selected qualifying medical records (e.g., each qualifying medical records within a lot/group of records), and for each selected qualifying medical record not having a pre-existing access-approval status, the managing entity may transmit (message to an app, or email, or text, etc.) a request for approval to the owner of the qualifying medical record, and update the approval status of the qualifying medical record in accordance with the owner's approval response.

Similarly, in response to the remote user specifying a desired number of qualifying medical records, the managing entity may check for a pre-existing access-approval status for each of the qualifying medical records. If there is a sufficient number of qualifying medical records having a pre-existing access-approval status, then the managing entity may select the desired number of qualifying medical records from among those having a pre-existing access-approval status. If there is not a sufficient number of qualifying medical records having a pre-existing access-approval status, the managing entity may then determine how many additional qualifying medical records are needed to meet the specified desired number, and for each additionally needed qualifying medical record, transmit a request for approval to the owner of the additionally needed qualifying medical record. The managing entity may then update the approval status of the additionally needed qualifying medical record in accordance with the owner's approval response.

Optionally, the managing entity may respond to the electronic request from the remote user for medical data meeting user-specified criteria, by providing the remote user with a price list associate with the listing of available qualifying medical records. The remote user may then choose to accept or reject the offered price. The managing entity may respond to the remote user selecting one or more of the qualifying medical records, by collecting the price associated with the qualifying medical records to which the remote user is granted access, and distributing a payment to one or more of each selected qualified medical record owner, medical institution that collected any of the qualifying medical records to which the remote user was granted access, data host that hosts any of the qualifying medical records to which the remote user was granted access, and/or the managing entity itself.

Although the managing entity may manage the above-described blockchain, the blockchain may additionally or alternatively be a public ledger computer network. Additionally, the received medical data may be, at least partly, stored on-chain (within the blockchain) and off-chain (external to the blockchain).

The managing entity may provide automatic data access to a doctor to which a patient (owner of the medical data) is being referred. For example, if the remote user is a doctor to which a patient is being referred to, and the user-specified criteria specifies medical records of the patient only, then the managing entity may maintain (or automatically set) the access-approval status for the qualifying medical records to indicate access approval.

Similarly, the managing entity may provide automatic data access to the owner of a medical record. For example, if the remote user is the owner of the medical data and the user-specified criteria specifies only medical records of the owner of the medical data, the managing entity maintains may maintain (automatic set) the access-approval status for the qualifying medical record to indicate access approval.

The managing entity may provide select users with free view access to the electronic ledger recorded in the blockchain, for transparency purposes. For example, the managing entity may maintain a group of privileged remote users, each of which has unimpeded review-access to the electronic ledger. The privileged users may be identified based on access-criteria established by the managing entity, such as one or more of prior given-authorization, the number of medical records submitted by the privileged users for storage to the managing entity, and prior agreed-upon subscription period, or paid subscription.

By way of example, the user-specified criteria submitted by the remote user may include one or more of a type of anatomical measurement, body function measurement, a data scan type, and an image type. For instance: the anatomical measurement may include eye pressure, keratometry measurements, refractive error, or eye size; the body function measurement includes an electrocardiogram, vital sign measurements (body temperature, pulse rate, respiration rate); the data scan type includes an A-scan, B-scan, or C-scan from an optical coherence tomography device; and the image type includes a fundus image, an en-face image, or an ophthalmic anterior segment image.

As stated above, the data transaction may optionally include some sort of incentive to motivate participation of data sharing. One example is to monetize the medical data transaction. For example, one may provide a method/system for using a computer to facilitate a transaction between a buyer and at least one seller, wherein the seller uses a private key to authorize submission of medical information to a managing entity (or data store). The managing entity may identify the seller by means of a public-key-based identifier that bears no relation to the seller's personal identity. Use of public-private key transactions are well-known and within the scope of those versed in the art. The buyer may submit a purchase offer for access to a specified type of medical information (e.g., meeting user-specified criteria) to the managing entity, and the managing entity may identify a listing of potential medical records (or sellers whose submitted medical information) matches the type of medical information specified in the purchase offer. In response to the seller using the seller's private key to accept the purchase offer (e.g., provide an access-approval status), the managing entity may complete the transaction, including providing the buyer with the requested access to the seller's medical information that matches the type of medical information specified in the purchase offer and providing payment to the seller and/or other stakeholders. The managing entity may maintain at least one of a description of the medical information submitted by the seller and the seller's public key using a blockchain computer network. The present transaction is then recorded on the blockchain. Optionally, the purchase offer may include an offer price, but the managing entity may provide a suggested purchase price to the buyer for the specified type of medical information. The buyer may accept the suggested purchase price or submit a different (higher or lower) purchase offer. Like in the above example, the seller may pre-approve the suggested purchase price prior to the seller submitting the purchase offer. In this case, the seller may pre-approve different purchase prices for different types of medical information found within the seller's submitted medical information (e.g., ophthalmic vs orthopedic, or image vs non-image, etc.).

Optionally, the managing entity may provide a machine model creation service, wherein the buyer's purchase offer may include a desired number of data samples of the specified type of medical information needed for the creation of the machine model. In this case, in response to collecting acceptances for the purchase offer from the sellers sufficient for meeting the desired number of data samples, the managing entity then creates the buyer-specified machine model using the data samples, and provides the created machine model to the buyer. In the case of selecting the machine model creation service, the buyer's requested access may exclude view access to the sellers' medical information. By way of example, the machine model may include a deep learning model selected from one or more of an artificial neural network, convolutional neural network, u-net, recurrent neural networks, generative adversarial networks, and multilayer perceptron's. Optionally, the machine model includes a machine learning model based one or more of a classification model, regression model, clustering, and dimensionality reduction.

In the above described transaction examples, the medical information (e.g., medical data/records) access provided to the remote user, or buyer, may exclude any information that personally identifies the data owner, or seller.

More generally, the present invention provides a method/system for sharing medical (e.g., ophthalmic) data. This method includes saving/storing the medical data to a blockchain. A remote user then requests access to the medical data stored/specified in the blockchain with a payment offer. The access request is then approved or rejected (for example, by the data owner). The access request decision is then recorded on the blockchain, and the remote user gets access to the medical data or receives a notice saying the request is rejected, based on the access request decision. Payment may then be provided to stakeholders when the access request is approved. The stakeholders can include the owner of the data, which may be the patient from whom the medical data was collected, doctor or medical facility that collected the data from the patient, data host that stores the data, and/or blockchain service provider (which may include the managing entity). Optionally, payment can be based on cryptocurrency. Additionally, the step of saving data to the blockchain can include saving both on-chain data and off-chain data. For example, on-chain data may include at least one of a data-owner identifier for contacting an owner of the (e.g., ophthalmic) data and storage location information indicating the location of any data stored off-chain. Preferably, approving or rejecting the access request is performed by the patient who owns the data. For example, approval or refusal of the access request may be received via an electronic (wired or wireless) communication device, such as from the patient from whom the medical data was collected. As is discussed above, access to the data can have multiple types, such as view only, rent with expiration time/period, permanent data download, and processing (e.g., only) within the network platform. In a processing-only access, the requested data is accessed and processed within a computer network platform remote from the remote user that requested the data access and may exclude view-access by the remote user.

The present invention also provides a method/system for referring a patient within a medical (e.g., ophthalmic) community. For example, this method may include saving patient medical data to the blockchain network; reviewing a medical data set by a first doctor and making a referral decision; sending the referral link to a referred second doctor in accordance with the referral decision; notifying the patient that his/her medical data is desired to be shared with a second doctor; the patient approving or rejecting the data sharing for this referral; recording the approval decision on the blockchain; and the second doctor getting access to the data or receiving notice saying the request is reject. Typically, a referral gets automatic access approval, as explained above, but in the present example, the patient can optionally halt the data sharing. In the present example, saving data to the blockchain can include saving both on-chain data and off-chain data. The referral link can be sent through email, text message, electronic messaging, etc. As before, approving or rejecting the access request is performed by the patient who owns the data.

The present invention also provides for a method/system for sharing medical (e.g., ophthalmic) data with a patient. The present method/system includes saving data to the blockchain network, and the blockchain can then authenticate the user accessing the data stored in the blockchain online. Saving data to the blockchain can include saving both on-chain data and off-chain data. Accessing the data stored in the blockchain online can be done through web browsers, mobile device apps, or computer applications.

The present invention further provides a method/system for building transparency and trust during medical (e.g., ophthalmic) data sharing. The method includes saving data to the blockchain network; storing all transactions on the blockchain (e.g., a networked public (transactions) ledger); and permitting members to access/view the public transactions ledger. Optionally, accessing the networked public transactions ledger by members requires certain criteria, such as a paid subscription.

Other objects and attainments together with a fuller understanding of the invention will become apparent and appreciated by referring to the following description and claims taken in conjunction with the accompanying drawings.

Several publications may be cited or referred to herein to facilitate the understanding of the present invention. All publications cited or referred to herein, are hereby incorporated herein in their entirety by reference.

The embodiments disclosed herein are only examples, and the scope of this disclosure is not limited to them. Any embodiment feature mentioned in one claim category, e.g. system, can be claimed in another claim category, e.g. method, as well. The dependencies or references back in the attached claims are chosen for formal reasons only. However, any subject matter resulting from a deliberate reference back to any previous claims can be claimed as well, so that any combination of claims and the features thereof are disclosed and can be claimed regardless of the dependencies chosen in the attached claims.

### BRIEF DESCRIPTION OF THE DRAWINGS

In the drawings wherein like reference symbols/characters refer to like parts:

FIG. 1 shows an exemplary blockchain computer network.

FIG. 2 provides a general overview an exemplary system in accord with the present invention.

FIG. 3 illustrates examples of some user-specified criteria for identifying desired medical data.

FIG. 4 shows examples a data count listing, of data-access types, and associated access prices.

FIG. 5 illustrates an exemplary data access transaction process between a remote user (e.g., data-customer or researcher) and the present managing entity (e.g., marketplace/workplace platform or blockchain administrator/service provider).

FIG. 6 illustrates a process for storing (moving) new patient data to the present managing entity (e.g., marketplace/workplace platform or blockchain administrator/service provider).

FIG. 7 illustrates a workflow for doctor referrals using the present system/platform.

FIG. 8 illustrates an example of a visual field test instrument (perimeter) for testing a patient's visual field.

FIG. 9 illustrates an example of a slit scanning ophthalmic system for imaging a fundus.

FIG. 10 illustrates a generalized frequency domain optical coherence tomography system used to collect 3D image data of the eye suitable for use with the present invention.

FIG. 11 shows an exemplary OCT B-scan image of a normal retina of a human eye, and illustratively identifies various canonical retinal layers and boundaries.

FIG. 12 shows an example of an enface vasculature image.

FIG. 13 shows an exemplary B-scan of a vasculature (OCTA) image.

FIG. 14 illustrates an example of a multilayer perceptron (MLP) neural network.

FIG. 15 shows a simplified neural network consisting of an input layer, a hidden layer, and an output layer.

FIG. 16 illustrates an example convolutional neural network architecture.

FIG. 17 illustrates an example U-Net architecture.

FIG. 18 illustrates an example computer system (or computing device or computer).

### DESCRIPTION OF THE PREFERRED EMBODIMENTS

Within the ophthalmic community (or other medical community), a typical process for sharing (e.g., medical) data between stakeholders (e.g., participants in a data transaction/exchange) may include the following steps:
1. Create a (e.g., medical) study proposal that lists the protocols (e.g., for gathering (medical) data, administering tests, selecting subjects, etc.);
2. Get IRB (e.g., Institutional Review Board or Ethics Committee) approval;
3. For each subject (e.g., patient in the medical study), get consent and generate the legal right (e.g., to use of the patient's medical data);
4. Acquire the needed data based on the protocols and save the data and consent to a database; and
5. Share all the acquired data/consents (e.g., with the stakeholders), such as through online sharing tools, external portable data-storage drives, etc.

However, there are several limitations to this current approach, including:
1) Obtaining consent is difficult, not scalable, and generally cannot be withdrawn. Generally, getting consent involves explaining technical details and clinical benefits to the patient, which can decrease the clinical workflow efficiency. Some patients will also decline the consent given limited time to understand the terms, limits, and purpose of the consent. Moreover, if the data is acquired under one specific use-case based on the consent, it may not be extended to other/future applications not covered in the original consent. Also, once the consent is given, it can be very difficult to withdraw the consent if the patient later changes his/her mind.
2) Establishing data sharing agreements with doctors is challenging. Quite often, doctors tend to limit data sharing to only close collaborators. For small companies and new researchers without well-established collaborators, getting study proposal or research agreements with doctors is difficult.
3) Patients and doctors do not have proper financial incentives for sharing data. Nowadays, data based innovative solutions can generate substantial economical revenues. However, patients, who are the owners of the (medical) data, generally do not get proper financial payment (or other compensation) for their data. This is also because current data sharing frameworks do not provide a mechanism for this to happen. Even for doctors who share the data, it is also difficult to obtain a good estimate of the value of the data due to a lack of free marketplace evaluation. This further imposes challenges when negotiating research proposals/contracts.
4) When all the study proposals and consents are done, the data sharing part itself is largely a manual process, which can make it very difficult to scale. This is particularly the case when shipping physical hard drives (or CDs, or DVDs, or flash drive, or other physical storage media) cross-country or globally. Such shipments raise risks of data loss during transportation.
5) In current data sharing frameworks, as the owner of the data, the patient usually cannot access his/her own data easily.

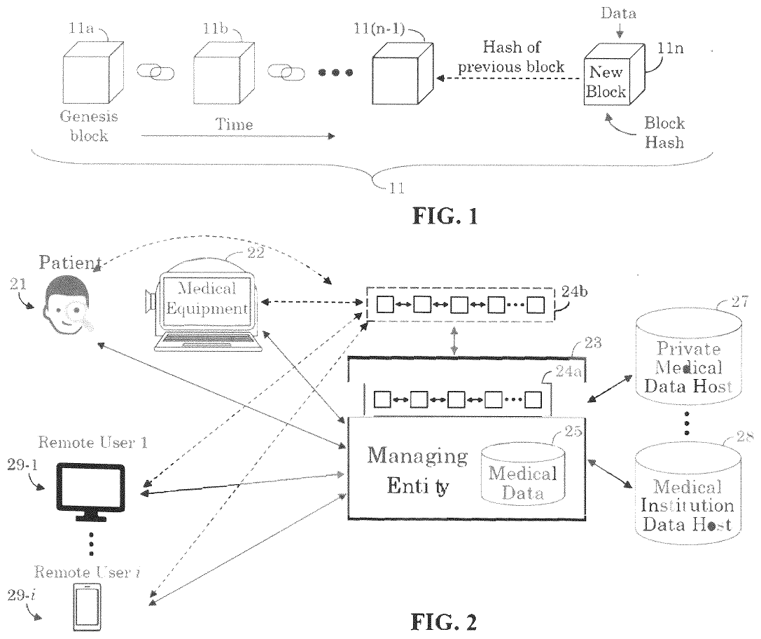

The present invention uses a blockchain technique to address the above issues. The construct of a typical blockchain computer network is well-known in the art, but for illustration purposes, FIG. 1 shows an exemplary blockchain computer network. Generally, a blockchain 11 is a distributed ledger with a growing list of records, or blocks [e.g., 11a, 11b, . . . , 11(n−1)] that are linked to each other using cryptography. The first block within a blockchain is typically termed the genesis block. Each block may contain a cryptographic hash of the block itself and the hash of the previous block (by which it identifies its position within the blockchain), a timestamp, and transaction data, but additional types of data may also be stored within a block. For example, a new block 11n contains data to be stored within it, the hash of the block 11n and the hash of the previous block 11(n−1). The data that is stored within a block depends on the type of the blockchain (e.g., the purpose of the blockchain). As it is known the art, a hash may be determined using a hashing algorithm, and provides a unique identifier that identifies a block and all of its contents. If the contents of a block are changed, a new hash will be generated to reflect this change. Thus, hashes can be used to determine when a block has been altered. Furthermore, the altered block's new hash effectively identifies it as a different block. Additionally, the timestamp can be used to ensure that the stored transaction data existed when the block was published and added to the blockchain. Since each block identifies (e.g., contain information about) the block previous to it, they form a chain, with each additional block reinforcing the ones before it. This makes blockchains resistant to modification of their data because once recorded, the data in any given block cannot be altered retroactively without also altering all subsequent blocks in the blockchain to reflect the alteration.

Previous ophthalmic blockchain applications do not target image data sharing. They are mostly focusing on the electronic medical record (EMR) aspect with mostly text data, which has limited value compared to diagnostic imaging data. Blockchain applications in other domains that allow image data to be shared do not provide a marketplace-based, image data commercialization system. In addition to other features of the present invention, the financial aspect of the present invention has the potential for a big impact in the industry and can create strong incentives to make data sharing substantiable, organic and patient centric. Moreover, the present method provides for multiple data-access types with optionally different price tiers, which provides flexibility to benefit different user groups. Data validation between on-chain data (data stored on the blockchain) and off-chain data (data stored off the blockchain, but the blockchain may optionally identify where the data is stored) may be provided to make sure the present blockchain-based solution fulfills any regulatory requirements and provides real world benefits. Transactions that occur within the blockchain are generally termed on-chain transactions and transactions that occur outside of the blockchain network are generally known as off-chain transactions. Regulatory requirements may also be addressed using digital (or “smart”) contracts in on-chain transactions and/or off-chain transaction. Additionally, referrals and patient access to their imaging data (at any time) may be handled differently in the present system than in prior approaches.

FIG. 2 provides a general overview an exemplary system in accord with the present invention. In the present example, a patient 21 may visit a private doctor or medical institution (e.g., clinic, hospital, research institute, etc.) that collects medical data from the patient 21 using medical equipment 22. The collected data may include answers to a patient medical questionnaire, patient's biological measurements, and/or medical images. Examples of different types of medical equipment include, but are not limited to, a visual field test instrument (perimeter) for testing a patient's visual field, a slit scanning ophthalmic system or other camera for imaging the anterior or posterior segments of an eye, an optical coherence tomography (OCT) system, and OCT angiography (OCTA) system. A more detailed discussion of some of these types of equipment is provided below. Examples of collected biological measurements and medical images include, are not limited to, retinal thickness maps, retinal layers, OCT/OCTA B-scans, OCT/OCTA A-scans, OCT/OCTA C-scans, OCT/OCTA enface images, images of specifically target biological/geographic features such the retina, macula, fovea, optic disc or nerve, posterior pole, specifically target vascular structures, the pupil, cornea, iris, etc. In the present example, the patient may grant permission (e.g., provide an agreement) prior to, or after the medical exam, for the doctor or medical institution to send all or part of the collected data to a managing entity (workplace platform or marketplace platform or blockchain service provider or blockchain administrator or data broker) 23. Optionally, the medical equipment/device that captures the medical measurements or images automatically sends/transmits the medical data to the managing entity 23, with consent from the patient such that there is no disturbance to the examination workflow. The data may be sent electronically via email, via the Internet (e.g., a web portal), computer application, portable device app, or on a physical data storage media (e.g., hard drive, CD, DVD, USB flash drive, etc.). Thus, with agreement of the owner of the medical data, the managing entity 23 receives the medical data for storage in a data store/host 25, or is otherwise granted permission to access the medical data from a remote data store/host 28. For example, the remote data store may be operated by the doctor or medical institution that collected the medical data. Optionally, the owner of the collected medial data, e.g., the patient in the present example, may directly communicate with the managing entity 23, and optionally directly submit his/her medical data to the managing entity for storage. Optionally, the data submission agreement with the managing entity 23 may grant the managing entity 23 free research access to the received medical data. Optionally, this research access may exclude personal identification information of a patient.

The managing entity 23 manages/oversees/governs and record transactions for the exchange of data (e.g., medical data/data records, which may include monetary transactions for data) between different stakeholders (e.g., patients, data owners, doctors, researchers, institutions, etc.). The managing entity 23 may hold/house a copy of the data, as illustrated by internal data store 25, or may access remotely held medical data such as illustrated by data store 27, which may be under management of the managing entity 23, or by independent data stores 28. For example, independent data store 28 may be managed by the doctor or institution that provided the data to the managing entity, or at least provided a description of the data including instructions for gaining access to its stored data.

The managing entity 23 may maintain hidden, e.g., secret, the personal identity of the data owners (and other stakeholders) before, during, and after a transaction, but also maintains an electronic ledger (e.g., electronic record) of all transactions including public identifiers of the involved stakeholders. As explained above, the managing entity 23 achieves this by use of one or more blockchains. The managing entity 23 thus records a summary of all transactions (e.g., the received medical data) to the blockchain, which may also maintain an electronic ledger of available medical records. The present managing entity 23 may host its own private blockchain 24a, and/or may maintain/manage a public blockchain 24b. In both cases, the management entity 23 may control who is given access to the blockchains 24a/24b. Optionally, the stakeholders may communication with private blockchain 24a via managing entity 23, as illustrated by solid arrows, or may be granted permission to communicate directly with public blockchain 24b, as illustrated by dotted arrows. The blockchain may function as a distributed ledger, which is akin to a database that can be consensually shared and synchronized across multiple sites, institutions, or geographies, accessible by multiple people, and can allows transactions to have public “witnesses,” which increases transparency. The participant at each node of the distributed public ledger network can be given access to the recorded transactions shared across that network. Any changes or additions (e.g., transactions) made to the ledger can thereby be reflected and verified by different stakeholders. The managing entity 23 may use decentralized (or public) identifiers (DIDs) that are associated with personal identifiable information (PII) and other private data. A DID may be public domain knowledge, but its associated PII remains hidden. Only the owner of a DID has access to its associated PII. In this manner, an owner can choose with whom to share private (e.g., medical) data, as well as when, where and how to share the private. This places access control of personal data on the blockchain with the owner of the personal data. Optionally, to speed up blockchain transactions, the managing entity 23 may execute a set of multiple transactions between the same stakeholders, and at the completion of the set of transactions, store a record of the transactions on the blockchain. Transactions that occur outside of the blockchain network, such as storing data to, or accessing data from, a data store off the blockchain are generally known as off-chain transactions, but a record of the off-chain transactions may also be maintained on the blockchain.

The blockchain 24a/24b may use a public key cryptosystem, which may be based on asymmetric encryption. In a public key cryptosystem, the network (e.g., managing entity 23 and/or blockchain 24a/24b) enables users to generate and use public-private key pairs, wherein a public key and its uniquely corresponding private key are generated together. The public key can be freely shared with anyone, but the private key functions as a secure passcode to unlock transactions belonging to its specifically paired public key. Therefore, private keys are typically be kept secret. In this manner, the public-private key pair enables secure transactions, such as communications and data exchanges. The transaction is encrypted using the public key, and requires the public key's corresponding private key to be decrypted. For example, if a first user wishes to receive a message from a second user, the first user would share their public key with the second user, and the second user would use the first user's public key to encrypt the message. Upon receiving the encrypted message, the first user may then use their own private key to decrypt the message. In all transactions, the users' personal identifying information remains hidden from public view.

Because public keys may be long strings of alpha-numeric characters, they can be unwieldy and cumbersome to use. Optionally, a modified representation of a public key may be used in place of the public key. This modified representation would preferably be shorter and easier to use than the original public key it represents. For instance, the modified representation of a public key in a public-private key pair may take the form of a “public address,” or public identifier. In particular implementations, the public address/identifier may take the form of a typical email address making it easier for users of the blockchain (e.g., stakeholders) to identify themselves to each other, since the public address can be freely shared. For example, a public address may be created using hashing algorithms on the public key it represents, which effectively adds extra layers of encryption. Thus, although a public address is typically easier to use than the public key it represents, it is still practically impossible to reverse-engineer a public address's corresponding private key. In the present example, when wanting to access the blockchain, one would present one's public address (or public key), but may also be required to validates one's identity by providing the public address's (e.g., the represented public key's) corresponding private key. If the private key is not correct, access to the blockchain is denied. In this manner, it can be assured that only the owner of a specific public address (or public key) is granted access.

Thus, the owner of the medical data stored/managed by the managing entity 23 may be identified by a public identifier, which may be based on a public key, that is associated with a corresponding personal identifiable information, which may be based on a private key. The public identifier preferably excludes any personal identifying information, whereby the managing entity 23 maintains hidden any personal identifying information of the owner of the medical data.

Multiple remote user, 29-1 to 29-i may transmit an electronic request for medical data meeting user-specified criteria. Optionally, the remote user, 29-1 to 29-i may review a ledger of available medical data (provided by the managing entity 23 and/or blockchain 24a/24b) prior to submitting the electronic request. After validating the request, as discussed above, the managing entity may respond to the electronic request by providing the remote user with a listing of available qualifying medical records that meet the user-specified criteria, as determined at least in part from the electronic ledger.

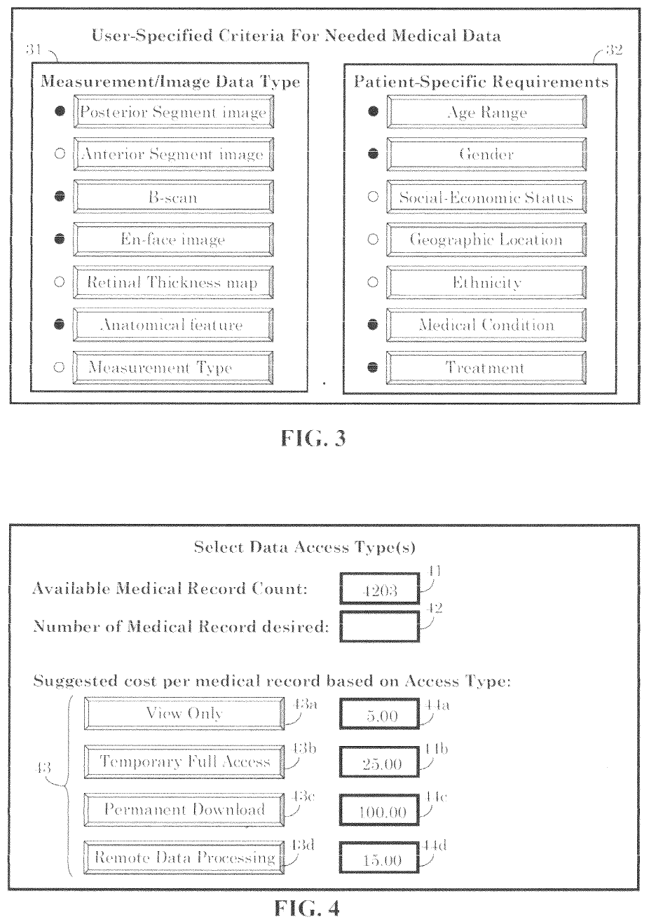

With reference to FIG. 3, examples of user-specified criteria may be divided by category. For example, the remote user 29-1 to 29-i may specify specific medical measurements or data types 31 and/or by patient-specific descriptors/requirements 32. The medical measurements or data types 31 may include anatomical measurement (such as eye pressure, keratometry measurements, refractive error, or eye size), body function measurement (e.g., electrocardiogram or vital sign measurements (body temperature, pulse rate, respiration rate)), data scan type (e.g., an A-scan, B-scan, or C-scan from an optical coherence tomography device, image type (e.g., fundus image, an en-face image, or ophthalmic anterior/posterior segment image), etc. The Patient-specific requirements may include age range, gender, social-economic status, geographic location (e.g., country, state, town, geographic region, etc.), ethnicity, existing medical condition (e.g., previously diagnosed illness), current treatment, etc.

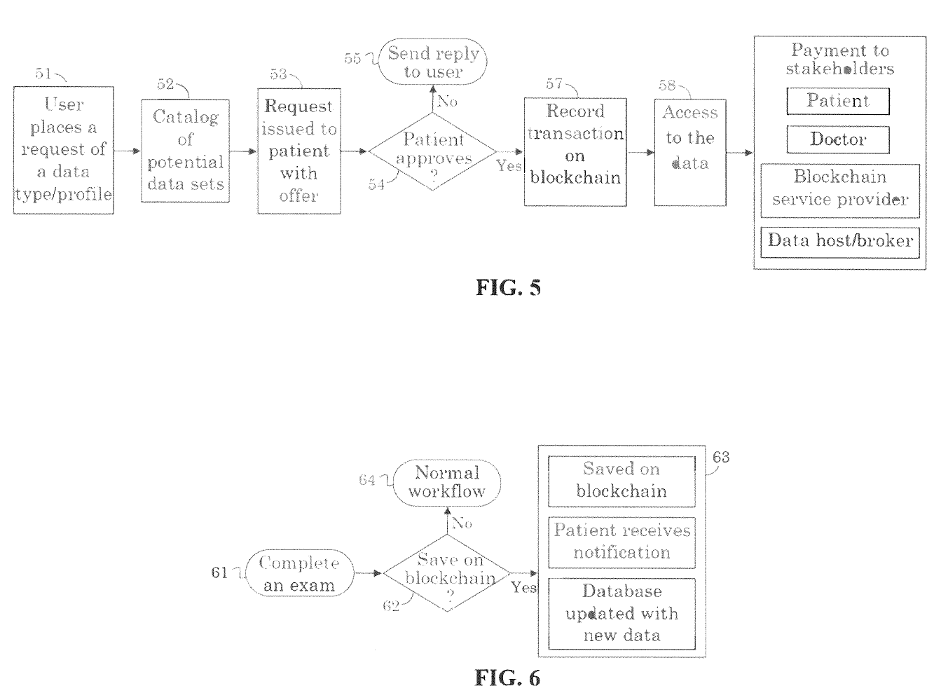

With reference to FIG. 4, optionally, the listing of available qualifying medical records that meet the user-specified criteria may include a count 41 of available (qualifying) medical records. If the count is not sufficient for the remote user's purposes, the remote user may choose to alter the user-specified criteria (see FIG. 3). The managing entity 23 may respond to this electronic altering of the user-specified criteria by automatically providing the remote user with an updated listing of available qualifying medical records that meet the altered user-specified criteria, including an update availability count 41.

The remote user may then select individual records and/or submit a general number of desired medical data records. In response to the remote user selecting one or more of the qualifying medical records, the managing entity 23 checks for a pre-existing access-approval status for each selected (qualifying) medical record. That is, the owner of individual medical data/records (e.g., the patient) may submit a pre-existing approval for access to their data in accord with certain criteria (e.g., access type, purpose of use, payment value, specific user requesting data, etc.). For each selected qualifying medical record that does not have a pre-existing access-approval status from the data owner, the managing entity transmits a request for approval (which may include a price offer) to the data owner and updates the approval status of the qualifying medical record in accordance with the data owner's approval response.

Optionally, the remote user may submit a desired number of qualifying medical records 42, which may be lower than the available record count 41. The managing entity 23 may respond by checking for the pre-existing access-approval status for each of the qualifying medical records, and if there is a sufficient number of qualifying medical records having a pre-existing access-approval status, the managing entity 23 may freely select the desired number of qualifying medical records from among those having a pre-existing access-approval status. However, if there is an insufficient number of qualifying medical records having a pre-existing access-approval status, the managing entity 23 may then determine how many additional qualifying medical records are needed to meet the specified desired number, and for each additionally needed qualifying medical record, transmit a request for approval to the owner of the additionally needed qualifying medical record, and update the approval status of the additionally needed qualifying medical record in accordance with the data owner's approval response.

In some cases, the managing entity 23 may assign an access-approval status to select medical data, in accord with pre-existing consent from the data owners. For example, if a first doctor is referring a patient to a second doctor, the managing entity 23 may grant the second doctor access to the patient's relevant medical records without needing a new access-approval response from the patient. For example, if the remote user is the second doctor to which the patient is being referred to, and the user-specified criteria specifies medical records of the patient only, the managing entity 23 maintains, or may assign, an automatic access-approval status for the qualifying medical records. In another instance, if a patient (or other data owner) wishes to access their own records, the request for a new access-approval response from the patient may be omitted. In other words, if the remote user that submits the request for medical data is the owner of the medical data and all the user-specified criteria specify only the medical records of the owner of the medical data, then the managing entity 23 may maintain, or may assign, an automatic access-approval status for the requested medical data.

As discussed above, the present system may provide a monetary incentive for stakeholder to participate in the present system. For example, the managing entity 23 may provide the remote user that requests medical data with a price list (e.g., per data item or per medical record) associated with the listing of available qualifying medical records. The price list may vary based on various criteria, such as the type of data being requested (e.g., measuring data, image data, questionnaire data, etc.), geographic location where the data was collected (e.g., country of origin, etc.), type of data access being requested, etc. Irrespective, if the remote user chooses to access any of the qualifying medical records, the managing entity 23 may collect from the remote user the price associated with the qualifying medical records, and distribute a payment to one or more of each of the selected medical record's owner, medical institution that collected any of the selected medical records, and data store that hosts any of the selected medical records, and to the managing entity itself. If the remote user does not accept the proffered price list, the remote user may submit a counteroffer (e.g., offer a different price) for the desired data, which may be higher or lower than that shown in the proffered price list. The managing entity 23 and/or data owner may then review the counteroffer and chose to accept or reject it. Future proffered price lists may be based, at least in part, on previously accepted counteroffers.

Irrespective of whether the managing entity 23 collects and distributes payments or provides some other type of incentive benefit, when the remote user selects one or more of the qualifying medical records for access, the managing entity 23 responds by granting the remote user access to the selected qualifying medical records in accordance with the access-approval status for each selected (qualifying) medical record granted by the owner of the selected (qualifying) medical record, and records the data transaction to the blockchain 24a/24b.

As discussed above, the managing entity 23 may provide different types of data access to a remote user. For instance with reference to FIG. 4, the managing entity 23 may receive from the remote user an access-type request selected from a list 43 of available access types for the data being requested. For example, the access-type list 43 may include data view only 43a, temporary (full) data access with expiration period 43b, permanent data access with data download 43c, and remote data processing 43d that is executed by (or executed under management of) the managing entity 23 remote from the remote user. More than one type of access request may be selected. For example, the Remote Data Processing option 43d may not include view-access. If the remote user wishes the option to review the data, then the remote user may additionally select the View Only option 43a. Optionally, The Permanent Download option 43b (e.g., permanent access with data download) may include removal of the accessed qualifying medical data from the listing of available medical records in the electronic ledger. If a monetary incentive is being implemented, then each access type may have an associated access-price 44a to 44d (e.g., per medical record value) payable by the remote user.

With remote data processing option 43d, the remote user further selects what type of data processing is desired. The managing entity 23 then executes, or arranges for execution at a remote site, the desired processing. For example, the desired data processing may include use of the selected medical data to generate of machine learning model (using the selected medical records) to address a specified objective, such locating the fovea in a fundus image. The remote user is then granted full access to the generated machine learning model. Examples of types of machine models include a deep learning model selected from one or more of an artificial neural network, convolutional neural network, u-net, recurrent neural networks, generative adversarial networks, and multilayer perceptron's. Examples of these types of machine models is provided below. More generally, the machine model option may additionally or alternatively include a machine learning model based one or more of a classification model, regression model, clustering, and dimensionality reduction.

As discussed above, the managing entity 23 may grant select (privileged) remote users access to the electronic ledger for review, which provides a record of available medical data for access. These privileged remote users may be granted unimpeded review-access to the electronic ledger. Privileged users may be identified based on access-criteria established by the managing entity 23, including one or more of prior given-authorization, the number of medical records submitted by the privileged users for storage to the managing entity 23, and/or a prior agreed-upon subscription-for-access price/period.

FIGS. 5 and 6 illustrate a first use case of the invention. The present embodiment illustrates a workflow for sharing (medical) data among blockchain network members (e.g., remote users, or stakeholders), e.g., through a marketplace. FIG. 5 illustrates data access (e.g., data exchange/purchase/lease) by a (remote) user (e.g., data-customer or researcher), and FIG. 6 illustrates a process for storing (moving) new patient (medical) data to the present marketplace platform (e.g., managing entity/blockchain). With reference to FIG. 5, first, a (remote) user of the blockchain places a request for a specific (medical) data type or profile (block 51). Based on the request, a catalog (e.g., listing) of available potential data sets that meet the request is aggregated (block 52), e.g., by the platform/managing entity. This aggregation can happen within the blockchain or done by a data broker that hosts the data sets (e.g., the managing entity). Based on this catalog, the corresponding requests for each of the data sets are issued to individual patients that own the requested data as one or more electronic notifications with an (e.g., price) offer based on the data-access type (block 53). The data-access type can include view only, rent/lease with expiration (period), permanent download, data-processing within the marketplace platform (e.g., the managing entity), etc. Different price tiers may be configured per data-access type so that the offer(s) varies based on the data-access type, as illustrated in FIG. 4.

The owner of the requested data (e.g., the patient) then has the choice to approve or reject data access (block 54). If the patient does not approve access, a corresponding reply, e.g., a notification of denied access, is sent to the (remote) user who submitted the access request (block 55). If the patient approves the access, which means the patient explicitly consents to sharing the patient's data, this explicit consent is then recorded on the blockchain and saved as permanent record (block 57). The user who requested access (e.g., the data-customer) is given access to the data in the format (data-access type) requested (block 58). A validation mechanism, as discussed above, is also built-in to the marketplace platform so that the data to which the (remote) user got access can be validated against the state/record when it was accessed/retrieved and optionally to when it was stored/saved to the platform/managing entity. If the validation passes, this data access is considered successful. Once access is completed, the payment transaction goes through, and all stakeholders get a proper portion of the payment (block 59). The proper portion of payment may be agreed upon prior to the transaction, or may be determined in real-time based on currently trending supply-and-demand values/percentages. For example, the patient who owns the data, the doctor/clinic who acquired the data, the hospital/imaging-center/device-companies who hosts the data, and the blockchain service all get payment. At any time, the patient can withdraw consent and a prorated payment will be refunded. After withdrawal of consent, any (previous) permanently downloaded data can no longer be used for research or commercial purposes.

The present marketplace platform may include various tools/services on the platform itself for purchased, leased, or otherwise accessed data. These tools may be used to help manipulate, analysis, or otherwise process the accessed data. For example, when processing of the accessed data is to be at least partially performed within the marketplace platform, the marketplace platform may allow the user (data-customer) to directly run one or more applications/tools/services (apps) on the purchased/leased data available via the blockchain. For instance, the marketplace platform may provide the data-customer access to one or more auto machine learning (ML) apps to help analyze the purchased/leased data. Various examples of ML architectures are provided below. Once a researcher (user) purchases/leases the data and chooses to process the purchased/leased data within the platform, the auto ML app can be purchased (or leased) and used on the purchased/leased data to generate a trained ML model, optionally following training instructions/configurations provided by the researcher. In another example, algorithms or analytic apps can be purchased/leased to run on the purchased/leased data in the blockchain to generate business insights.

With reference to FIG. 6, new patient data saving (storing) to the blockchain (managing entity) may be implemented in the background and be fully compatible with existing clinical workflows. For example, after a medical exam is completed (block 61), based on the patient's preference/permissions, the patient's data can be saved/transmitted to the blockchain (managing entity) or not (block 62). If the patient prefers not to opt-in to the marketplace platform (block 62=No), the (doctor's/clinic's) normal workflow is followed, and no patient data is moved to the workplace platform (block 64). If the patient's preference is to opt-in to the marketplace platform (block 62=Yes), the patient's exam data is saved to the blockchain (managing entity/platform), and the patient will receive a notification after successful data storage (block 63). At the same time, this patient data (e.g., image data set) is added to a data sharing marketplace database (e.g., electronic ledger/blockchain), as indicated by block 63.

In a similar way as sharing patient/medical data, algorithms/applications can be shared using the blockchain (managing entity/platform) too. In this case, the algorithm/application is processed in a manner similar to how the “data” is processed, and can be tested/licensed/purchased in the marketplace platform using the blockchain. In this manner, all the approval and transaction records are saved on the blockchain automatically.

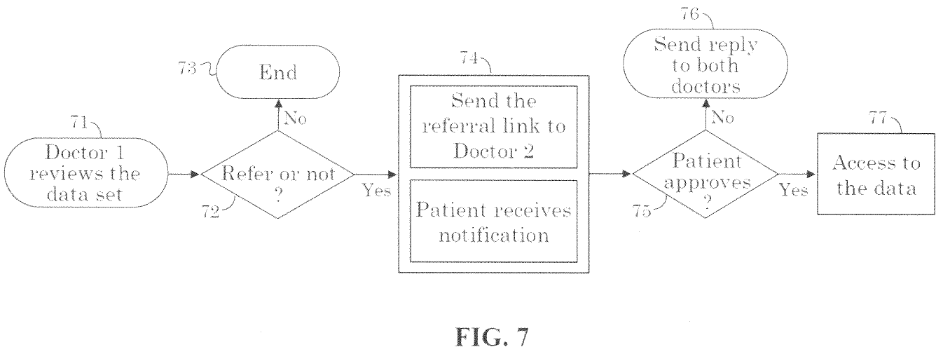

FIG. 7 illustrates a workflow for doctor referrals using the present managing entity/platform (e.g., using a blockchain). In this use case, a first doctor reviews the patient data (block 71) and then decides whether to refer the patient to a second doctor or not (block 72). If the referral decision is no (block 72=No), the process ends (block 73). If the referral decision is yes (block 72=Yes), a referral link (e.g., email, electronic message, URL, etc.) will be sent to the second doctor with instructions on how to join the blockchain network (block 74), e.g., how to register as a user with the managing entity. At the same time, the patient who owns the data may also receive a notification indicting that his/her medical data/record will be shared with the second doctor (block 74), e.g., as part of the doctor referral. In one embodiment, the patient can then approve or disapprove the sharing of his/her data (block 75). If the patient disapproves (block 75=No), both doctors receive a reply indicating the patient's refusal to have his/her data shared with the second doctor (block 76). This would be similar to the patient refusing the referral. If the patient approves the sharing of his/her data (block 75=Yes), the second doctor gains access to the patient's data (block 77). Alternatively, this patient approval process can be integrated into the clinical workflow as part of a patient-agreement to allow the patient's data to be shared with other doctors (automatically).

Optionally, the patient whose data is saved on the blockchain network can access his/her own data from a web portal (e.g. web browser) or app at any time, as discussed above. Once the patient's credential is authenticated, the patient has full consent to access his/her own data, by default.

Optimally, the marketplace platform (managing entity) may provide for members of the blockchain network who meet certain requirements (e.g., subscription paid, meet a minimum number of data sets shared) to have access to the blockchain ledger summary information that has all the recorded block information in it. This would provide transparency in the blockchain network so that trust may be built by everyone having access to the single one true record.

Hereinafter is provided a description of various hardware and architectures suitable for the present invention.

### Visual Field Test System

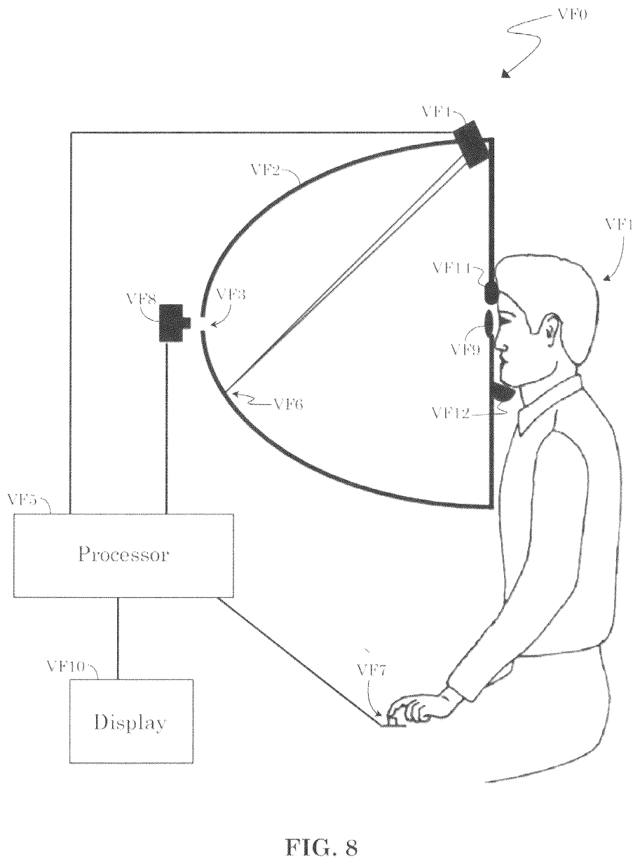

The improvements described herein may be used in conjunction with any type of visual field tester/system, e.g., perimeter. One such system is a “bowl” visual field tester VF0, as illustrated in FIG. 8. A subject (e.g., patient) VF1 is shown observing a hemispherical projection screen (or other type of display) VF2 generally shaped as a bowl, for which the tester VF0 is so termed. Typically, the subject is instructed to fixate at a point at the center of the hemispherical screen VF3. The subject rests his/her head on a patient support, which may include a chin rest VF12 and/or a forehead rest VF14. For instance, the subject rests his/her head on the chin rest VF12 and places his/her forehead against the forehead rest VF14. Optionally, the chin rest VF12 and the forehead rest VF14 may be moved together or independently of one another to correctly fixate/position the patient's eye, e.g., relative to a trial lens holder VF9 that may hold a lens through which the subject may view screen VF2. For example, the chin rest and headrest may move independently in the vertical direction to accommodate different patient head sizes and move together in the horizontal and/or vertical direction to correctly position the head. However, this is not limiting, and other arrangements/movements can be envisioned by one skilled in the art.

A projector, or other imaging device, VF4 under control of a processor VF5 displays a series of test stimuli (e.g., test points of any shape) VF6 onto the screen VF2. The subject VF1 indicates that he/she sees a stimulus VF6 by actuating a user input VF7 (e.g., depressing an input button). This subject response may be recorded by processor VF5, which may function to evaluate the visual field of an eye based on the subject's responses, e.g., determine the size, position, and/or intensity of a test stimulus VF6 at which it can no longer be seen by the subject VF1, and thereby determine the (visible) threshold of the test stimulus VF6. A camera VF8 may be used to capture the gaze (e.g., gaze direction) of the patient throughout the test. Gaze direction may be used for patient alignment and/or to ascertain the patient's adherence to proper test procedures. In the present example, the camera VF8 is located on the Z-axis relative to the patient's eye (e.g. relative to trial lens holder VF9) and behind the bowl (of screen VF2) for capturing live images(s) or video of the patient's eye. In other embodiments, this camera may be located off this Z-axis. The images from the gaze camera VF8 can optionally be displayed on a second display VF10 to a clinician (who may also be interchangeably referred to herein as a technician) for aid in patient alignment or test verification. The camera VF8 may record and store one or more images of the eye during each stimulus presentation. This may lead to a collection of anywhere from tens to hundreds of images per visual field test, depending on the testing conditions. Alternatively, the camera VF8 may record and store a full length movie during the test and provide time stamps indicating when each stimulus is presented. Additionally, images may also be collected between stimulus presentations to provide details on the subject's overall attention throughout the VF test's duration.

Trial lens holder VF9 may be positioned in front of the patient's eye to correct for any refractive error in the eye. Optionally, the lens holder VF9 may carry or hold a liquid trial lens (see for example U.S. Pat. No. 8,668,338, the contents of which are hereby incorporated in their entirety by reference), which may be utilized to provide variable refractive correction for the patient VF1. However, it should be noted that the present invention is not limited to using a liquid trial lens for refraction correction and other conventional/standard trial lenses known in the art may also be used.

In some embodiments, one or more light sources (not shown) may be positioned in front of the eye of the subject VF1, which create reflections from ocular surfaces such as the cornea. In one variation, the light sources may be light-emitting diodes (LEDs).

While FIG. 8 shows a projection type visual field tester VF0, the invention described herein may be used with other types of devices (visual field testers), including those that generate images through a liquid crystal display (LCD) or other electronic display (see for example U.S. Pat. No. 8,132,916, hereby incorporated by reference). Other types of visual field testers include, for example, flat-screen testers, miniaturized testers, and binocular visual field testers. Examples of these types of testers may be found in U.S. Pat. Nos. 8,371,696, 5,912,723, 8,931,905, US designed patent D472637, each of which is hereby incorporated in its entirety by reference.

Visual field tester VF0 may incorporate an instrument-control system (e.g. running an algorithm, which may be software, code, and/or routine) that uses hardware signals and a motorized positioning system to automatically position the patient's eye at a desired position, e.g., the center of a refraction correction lens at lens holder VF9. For example, stepper motors may move chin rest VF12 and the forehead rest VF14 under software control. A rocker switch may be provided to enable the attending technician to adjust the patient's head position by causing the chin rest and forehead stepper motors to operate. A manually moveable refraction lens may also be placed in front of the patient's eye on lens holder VF9 as close to the patient's eye as possible without adversely affecting the patient's comfort. Optionally, the instrument control algorithm may pause perimetry test execution while chin rest and/or forehead motor movements are under way if such movements would disrupt test execution.

### Fundus Imaging System

Two categories of imaging systems used to image the fundus are flood illumination imaging systems (or flood illumination imagers) and scan illumination imaging systems (or scan imagers). Flood illumination imagers flood with light an entire field of view (FOV) of interest of a specimen at the same time, such as by use of a flash lamp, and capture a full-frame image of the specimen (e.g., the fundus) with a full-frame camera (e.g., a camera having a two-dimensional (2D) photo sensor array of sufficient size to capture the desired FOV, as a whole). For example, a flood illumination fundus imager would flood the fundus of an eye with light, and capture a full-frame image of the fundus in a single image capture sequence of the camera. A scan imager provides a scan beam that is scanned across a subject, e.g., an eye, and the scan beam is imaged at different scan positions as it is scanned across the subject creating a series of image-segments that may be reconstructed, e.g., montaged, to create a composite image of the desired FOV. The scan beam could be a point, a line, or a two-dimensional area such a slit or broad line. Examples of fundus imagers are provided in U.S. Pat. Nos. 8,967,806 and 8,998,411.

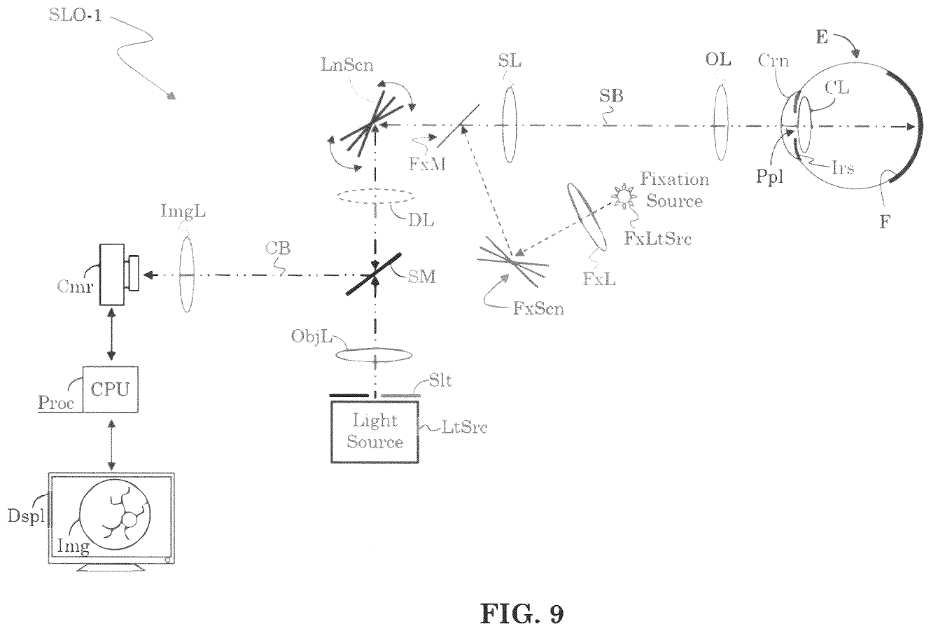

FIG. 9 illustrates an example of a slit scanning ophthalmic system SLO-1 for imaging a fundus F, which is the interior surface of an eye E opposite the eye lens (or crystalline lens) CL and may include the retina, optic disc, macula, fovea, and posterior pole. In the present example, the imaging system is in a so-called “scan-descan” configuration, wherein a scanning line beam SB traverses the optical components of the eye E (including the cornea Cm, iris Irs, pupil Ppl, and crystalline lens CL) to be scanned across the fundus F. In the case of a flood fundus imager, no scanner is needed, and the light is applied across the entire, desired field of view (FOV) at once. Other scanning configurations are known in the art, and the specific scanning configuration is not critical to the present invention. As depicted, the imaging system includes one or more light sources LtSrc, preferably a multi-color LED system or a laser system in which the etendue has been suitably adjusted. An optional slit Slt (adjustable or static) is positioned in front of the light source LtSrc and may be used to adjust the width of the scanning line beam SB. Additionally, slit Slt may remain static during imaging or may be adjusted to different widths to allow for different confocality levels and different applications either for a particular scan or during the scan for use in suppressing reflexes. An optional objective lens ObjL may be placed in front of the slit Slt. The objective lens ObjL can be any one of state-of-the-art lenses including but not limited to refractive, diffractive, reflective, or hybrid lenses/systems. The light from slit Slt passes through a pupil splitting mirror SM and is directed towards a scanner LnScn. It is desirable to bring the scanning plane and the pupil plane as near together as possible to reduce vignetting in the system. Optional optics DL may be included to manipulate the optical distance between the images of the two components. Pupil splitting mirror SM may pass an illumination beam from light source LtSrc to scanner LnScn, and reflect a detection beam from scanner LnScn (e.g., reflected light returning from eye E) toward a camera Cmr. A task of the pupil splitting mirror SM is to split the illumination and detection beams and to aid in the suppression of system reflexes. The scanner LnScn could be a rotating galvo scanner or other types of scanners (e.g., piezo or voice coil, micro-electromechanical system (MEMS) scanners, electro-optical deflectors, and/or rotating polygon scanners). Depending on whether the pupil splitting is done before or after the scanner LnScn, the scanning could be broken into two steps wherein one scanner is in an illumination path and a separate scanner is in a detection path. Specific pupil splitting arrangements are described in detail in U.S. Pat. No. 9,456,746, which is herein incorporated in its entirety by reference.

From the scanner LnScn, the illumination beam passes through one or more optics, in this case a scanning lens SL and an ophthalmic or ocular lens OL, that allow for the pupil of the eye E to be imaged to an image pupil of the system. Generally, the scan lens SL receives a scanning illumination beam from the scanner LnScn at any of multiple scan angles (incident angles), and produces scanning line beam SB with a substantially flat surface focal plane (e.g., a collimated light path). Ophthalmic lens OL may then focus the scanning line beam SB onto an object to be imaged. In the present example, ophthalmic lens OL focuses the scanning line beam SB onto the fundus F (or retina) of eye E to image the fundus. In this manner, scanning line beam SB creates a traversing scan line that travels across the fundus F. One possible configuration for these optics is a Kepler type telescope wherein the distance between the two lenses is selected to create an approximately telecentric intermediate fundus image (4-f configuration). The ophthalmic lens OL could be a single lens, an achromatic lens, or an arrangement of different lenses. All lenses could be refractive, diffractive, reflective or hybrid as known to one skilled in the art. The focal length(s) of the ophthalmic lens OL, scan lens SL and the size and/or form of the pupil splitting mirror SM and scanner LnScn could be different depending on the desired field of view (FOV), and so an arrangement in which multiple components can be switched in and out of the beam path, for example by using a flip in optic, a motorized wheel, or a detachable optical element, depending on the field of view can be envisioned. Since the field of view change results in a different beam size on the pupil, the pupil splitting can also be changed in conjunction with the change to the FOV. For example, a 450 to 600 field of view is a typical, or standard, FOV for fundus cameras. Higher fields of view, e.g., a widefield FOV, of 60°-120°, or more, may also be feasible. A widefield FOV may be desired for a combination of the Broad-Line Fundus Imager (BLFI) with another imaging modalities such as optical coherence tomography (OCT). The upper limit for the field of view may be determined by the accessible working distance in combination with the physiological conditions around the human eye. Because a typical human retina has a FOV of 140° horizontal and 80°-100° vertical, it may be desirable to have an asymmetrical field of view for the highest possible FOV on the system.

The scanning line beam SB passes through the pupil Ppl of the eye E and is directed towards the retinal, or fundus, surface F. The scanner LnScn1 adjusts the location of the light on the retina, or fundus, F such that a range of transverse locations on the eye E are illuminated. Reflected or scattered light (or emitted light in the case of fluorescence imaging) is directed back along as similar path as the illumination to define a collection beam CB on a detection path to camera Cmr.

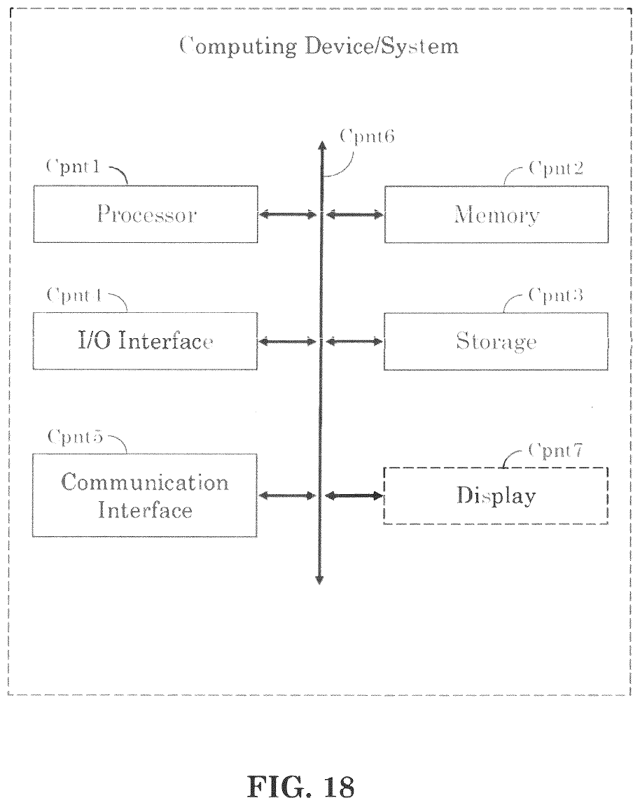

In the “scan-descan” configuration of the present, exemplary slit scanning ophthalmic system SLO-1, light returning from the eye E is “descanned” by scanner LnScn on its way to pupil splitting mirror SM. That is, scanner LnScn scans the illumination beam from pupil splitting mirror SM to define the scanning illumination beam SB across eye E, but since scanner LnScn also receives returning light from eye E at the same scan position, scanner LnScn has the effect of descanning the returning light (e.g., cancelling the scanning action) to define a non-scanning (e.g., steady or stationary) collection beam from scanner LnScn to pupil splitting mirror SM, which folds the collection beam toward camera Cmr. At the pupil splitting mirror SM, the reflected light (or emitted light in the case of fluorescence imaging) is separated from the illumination light onto the detection path directed towards camera Cmr, which may be a digital camera having a photo sensor to capture an image. An imaging (e.g., objective) lens ImgL may be positioned in the detection path to image the fundus to the camera Cmr. As is the case for objective lens ObjL, imaging lens ImgL may be any type of lens known in the art (e.g., refractive, diffractive, reflective or hybrid lens). Additional operational details, in particular, ways to reduce artifacts in images, are described in PCT Publication No. WO2016/124644, the contents of which are herein incorporated in their entirety by reference. The camera Cmr captures the received image, e.g., it creates an image file, which can be further processed by one or more (electronic) processors or computing devices (e.g., the computer system of FIG. 18). Thus, the collection beam (returning from all scan positions of the scanning line beam SB) is collected by the camera Cmr, and a full-frame image Img may be constructed from a composite of the individually captured collection beams, such as by montaging. However, other scanning configuration are also contemplated, including ones where the illumination beam is scanned across the eye E and the collection beam is scanned across a photo sensor array of the camera. PCT Publication WO 2012/059236 and US Patent Publication No. 2015/0131050, herein incorporated by reference, describe several embodiments of slit scanning ophthalmoscopes including various designs where the returning light is swept across the camera's photo sensor array and where the returning light is not swept across the camera's photo sensor array.

In the present example, the camera Cmr is connected to a processor (e.g., processing module) Proc and a display (e.g., displaying module, computer screen, electronic screen, etc.) Dspl, both of which can be part of the image system itself, or may be part of separate, dedicated processing and/or displaying unit(s), such as a computer system wherein data is passed from the camera Cmr to the computer system over a cable or computer network including wireless networks. The display and processor can be an all in one unit. The display can be a traditional electronic display/screen or of the touch screen type and can include a user interface for displaying information to and receiving information from an instrument operator, or user. The user can interact with the display using any type of user input device as known in the art including, but not limited to, mouse, knobs, buttons, pointer, and touch screen.

It may be desirable for a patient's gaze to remain fixed while imaging is carried out. One way to achieve this is to provide a fixation target that the patient can be directed to stare at. Fixation targets can be internal or external to the instrument depending on what area of the eye is to be imaged. One embodiment of an internal fixation target is shown in FIG. 9. In addition to the primary light source LtSrc used for imaging, a second optional light source FxLtSrc, such as one or more LEDs, can be positioned such that a light pattern is imaged to the retina using lens FxL, scanning element FxScn and reflector/mirror FxM. Fixation scanner FxScn can move the position of the light pattern and reflector FxM directs the light pattern from fixation scanner FxScn to the fundus F of eye E. Preferably, fixation scanner FxScn is position such that it is located at the pupil plane of the system so that the light pattern on the retina/fundus can be moved depending on the desired fixation location.

Slit-scanning ophthalmoscope systems are capable of operating in different imaging modes depending on the light source and wavelength selective filtering elements employed. True color reflectance imaging (imaging similar to that observed by the clinician when examining the eye using a hand-held or slit lamp ophthalmoscope) can be achieved when imaging the eye with a sequence of colored LEDs (red, blue, and green). Images of each color can be built up in steps with each LED turned on at each scanning position or each color image can be taken in its entirety separately. The three, color images can be combined to display the true color image, or they can be displayed individually to highlight different features of the retina. The red channel best highlights the choroid, the green channel highlights the retina, and the blue channel highlights the anterior retinal layers. Additionally, light at specific frequencies (e.g., individual colored LEDs or lasers) can be used to excite different fluorophores in the eye (e.g., autofluorescence) and the resulting fluorescence can be detected by filtering out the excitation wavelength.

The fundus imaging system can also provide an infrared reflectance image, such as by using an infrared laser (or other infrared light source). The infrared (IR) mode is advantageous in that the eye is not sensitive to the IR wavelengths. This may permit a user to continuously take images without disturbing the eye (e.g., in a preview/alignment mode) to aid the user during alignment of the instrument. Also, the IR wavelengths have increased penetration through tissue and may provide improved visualization of choroidal structures. In addition, fluorescein angiography (FA) and indocyanine green (ICG) angiography imaging can be accomplished by collecting images after a fluorescent dye has been injected into the subject's bloodstream. For example, in FA (and/or ICG) a series of time-lapse images may be captured after injecting a light-reactive dye (e.g., fluorescent dye) into a subject's bloodstream. It is noted that care must be taken since the fluorescent dye may lead to a life-threatening allergic reaction in a portion of the population. High contrast, greyscale images are captured using specific light frequencies selected to excite the dye. As the dye flows through the eye, various portions of the eye are made to glow brightly (e.g., fluoresce), making it possible to discern the progress of the dye, and hence the blood flow, through the eye.

### Optical Coherence Tomography Imaging System

Generally, optical coherence tomography (OCT) uses low-coherence light to produce two-dimensional (2D) and three-dimensional (3D) internal views of biological tissue. OCT enables in vivo imaging of retinal structures. OCT angiography (OCTA) produces flow information, such as vascular flow from within the retina. Examples of OCT systems are provided in U.S. Pat. Nos. 6,741,359 and 9,706,915, and examples of an OCTA systems may be found in U.S. Pat. Nos. 9,700,206 and 9,759,544, all of which are herein incorporated in their entirety by reference. An exemplary OCT/OCTA system is provided herein.

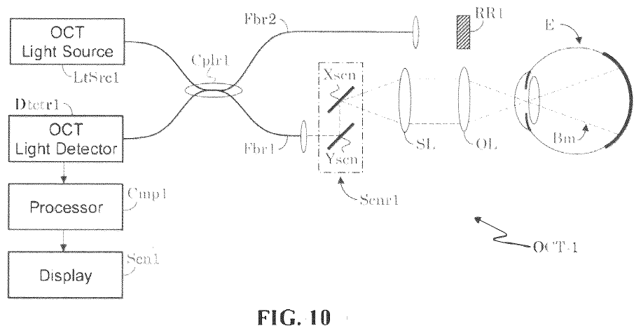

FIG. 10 illustrates a generalized frequency domain optical coherence tomography (FD-OCT) system used to collect 3D image data of the eye suitable for use with the present invention. An FD-OCT system OCT_1 includes a light source, LtSrc1. Typical light sources include, but are not limited to, broadband light sources with short temporal coherence lengths or swept laser sources. A beam of light from light source LtSrc1 is routed, typically by optical fiber Fbr1, to illuminate a sample, e.g., eye E; a typical sample being tissues in the human eye. The light source LrSrc1 may, for example, be a broadband light source with short temporal coherence length in the case of spectral domain OCT (SD-OCT) or a wavelength tunable laser source in the case of swept source OCT (SS-OCT). The light may be scanned, typically with a scanner Scnr1 between the output of the optical fiber Fbr1 and the sample E, so that the beam of light (dashed line Bm) is scanned laterally over the region of the sample to be imaged. The light beam from scanner Scnr1 may pass through a scan lens SL and an ophthalmic lens OL and be focused onto the sample E being imaged. The scan lens SL may receive the beam of light from the scanner Scnr1 at multiple incident angles and produces substantially collimated light, ophthalmic lens OL may then focus onto the sample. The present example illustrates a scan beam that needs to be scanned in two lateral directions (e.g., in x and y directions on a Cartesian plane) to scan a desired field of view (FOV). An example of this would be a point-field OCT, which uses a point-field beam to scan across a sample. Consequently, scanner Scnr1 is illustratively shown to include two sub-scanner: a first sub-scanner Xscn for scanning the point-field beam across the sample in a first direction (e.g., a horizontal x-direction); and a second sub-scanner Yscn for scanning the point-field beam on the sample in traversing second direction (e.g., a vertical y-direction). If the scan beam were a line-field beam (e.g., a line-field OCT), which may sample an entire line-portion of the sample at a time, then only one scanner may be needed to scan the line-field beam across the sample to span the desired FOV. If the scan beam were a full-field beam (e.g., a full-field OCT), no scanner may be needed, and the full-field light beam may be applied across the entire, desired FOV at once.

Irrespective of the type of beam used, light scattered from the sample (e.g., sample light) is collected. In the present example, scattered light returning from the sample is collected into the same optical fiber Fbr1 used to route the light for illumination. Reference light derived from the same light source LtSrc1 travels a separate path, in this case involving optical fiber Fbr2 and retro-reflector RR1 with an adjustable optical delay. Those skilled in the art will recognize that a transmissive reference path can also be used and that the adjustable delay could be placed in the sample or reference arm of the interferometer. Collected sample light is combined with reference light, for example, in a fiber coupler Cplr1, to form light interference in an OCT light detector Dtctr1 (e.g., photodetector array, digital camera, etc.). Although a single fiber port is shown going to the detector Dtctr1, those skilled in the art will recognize that various designs of interferometers can be used for balanced or unbalanced detection of the interference signal. The output from the detector Dtctr1 is supplied to a processor (e.g., internal or external computing device) Cmp1 that converts the observed interference into depth information of the sample. The depth information may be stored in a memory associated with the processor Cmp1 and/or displayed on a display (e.g., computer/electronic display/screen) Scn1. The processing and storing functions may be localized within the OCT instrument, or functions may be offloaded onto (e.g., performed on) an external processor (e.g., an external computing device), to which the collected data may be transferred. An example of a computing device (or computer system) is shown in FIG. 18. This unit could be dedicated to data processing or perform other tasks which are quite general and not dedicated to the OCT device. The processor (computing device) Cmp1 may include, for example, a field-programmable gate array (FPGA), a digital signal processor (DSP), an application specific integrated circuit (ASIC), a graphics processing unit (GPU), a system on chip (SoC), a central processing unit (CPU), a general purpose graphics processing unit (GPGPU), or a combination thereof, that may performs some, or the entire, processing steps in a serial and/or parallelized fashion with one or more host processors and/or one or more external computing devices.

The sample and reference arms in the interferometer could consist of bulk-optics, fiber-optics, or hybrid bulk-optic systems and could have different architectures such as Michelson, Mach-Zehnder or common-path based designs as would be known by those skilled in the art. Light beam as used herein should be interpreted as any carefully directed light path. Instead of mechanically scanning the beam, a field of light can illuminate a one or two-dimensional area of the retina to generate the OCT data (see for example, U.S. Pat. No. 9,332,902; D. Hillmann et al, “Holoscopy—Holographic Optical Coherence Tomography,” Optics Letters, 36(13): 2390 2011; Y. Nakamura, et al, “High-Speed Three Dimensional Human Retinal Imaging by Line Field Spectral Domain Optical Coherence Tomography,” Optics Express, 15(12):7103 2007; Blazkiewicz et al, “Signal-To-Noise Ratio Study of Full-Field Fourier-Domain Optical Coherence Tomography,” Applied Optics, 44(36):7722 (2005)). In time-domain systems, the reference arm needs to have a tunable optical delay to generate interference. Balanced detection systems are typically used in TD-OCT and SS-OCT systems, while spectrometers are used at the detection port for SD-OCT systems. The invention described herein could be applied to any type of OCT system. Various aspects of the invention could apply to any type of OCT system or other types of ophthalmic diagnostic systems and/or multiple ophthalmic diagnostic systems including but not limited to fundus imaging systems, visual field test devices, and scanning laser polarimeters.

In Fourier Domain optical coherence tomography (FD-OCT), each measurement is the real-valued spectral interferogram (Sj(k)). The real-valued spectral data typically goes through several post-processing steps including background subtraction, dispersion correction, etc. The Fourier transform of the processed interferogram, results in a complex valued OCT signal output Aj(z)=|Aj|eiφ, The absolute value of this complex OCT signal, |Aj|, reveals the profile of scattering intensities at different path lengths, and therefore scattering as a function of depth (z-direction) in the sample. Similarly, the phase, φj can also be extracted from the complex valued OCT signal. The profile of scattering as a function of depth is called an axial scan (A-scan). A set of A-scans measured at neighboring locations in the sample produces a cross-sectional image (tomogram or B-scan) of the sample. A collection of B-scans collected at different transverse locations on the sample makes up a data volume or cube. For a particular volume of data, the term fast axis refers to the scan direction along a single B-scan whereas slow axis refers to the axis along which multiple B-scans are collected. The term “cluster scan” may refer to a single unit or block of data generated by repeated acquisitions at the same (or substantially the same) location (or region) for the purposes of analyzing motion contrast, which may be used to identify blood flow. A cluster scan can consist of multiple A-scans or B-scans collected with relatively short time separations at approximately the same location(s) on the sample. Since the scans in a cluster scan are of the same region, static structures remain relatively unchanged from scan to scan within the cluster scan, whereas motion contrast between the scans that meets predefined criteria may be identified as blood flow.

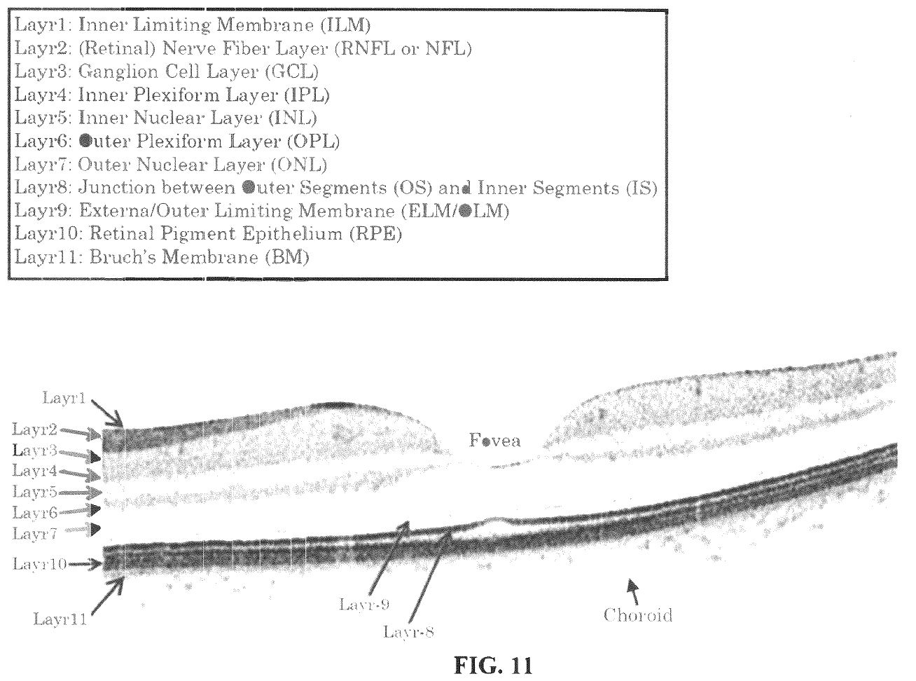

A variety of ways to create B-scans are known in the art including but not limited to: along the horizontal or x-direction, along the vertical or y-direction, along the diagonal of x and y, or in a circular or spiral pattern. B-scans may be in the x-z dimensions but may be any cross-sectional image that includes the z-dimension. An example OCT B-scan image of a normal retina of a human eye is illustrated in FIG. 11. An OCT B-scan of the retinal provides a view of the structure of retinal tissue. For illustration purposes, FIG. 11 identifies various canonical retinal layers and layer boundaries. The identified retinal boundary layers include (from top to bottom): the inner limiting membrane (TLM) Lyer1, the retinal nerve fiber layer (RNFL or NFL) Layr2, the ganglion cell layer (GCL) Layr3, the inner plexiform layer (IPL) Layr4, the inner nuclear layer (INL) Layr5, the outer plexiform layer (OPL) Layr6, the outer nuclear layer (ONL) Layr7, the junction between the outer segments (OS) and inner segments (IS) (indicated by reference character Layr8) of the photoreceptors, the external or outer limiting membrane (ELM or OLM) Layr9, the retinal pigment epithelium (RPE) Layr10, and the Bruch's membrane (BM) Layr11.

In OCT Angiography, or Functional OCT, analysis algorithms may be applied to OCT data collected at the same, or approximately the same, sample locations on a sample at different times (e.g., a cluster scan) to analyze motion or flow (see for example US Patent Publication Nos. 2005/0171438, 2012/0307014, 2010/0027857, 2012/0277579 and U.S. Pat. No. 6,549,801, all of which are herein incorporated in their entirety by reference). An OCT system may use any one of a number of OCT angiography processing algorithms (e.g., motion contrast algorithms) to identify blood flow. For example, motion contrast algorithms can be applied to the intensity information derived from the image data (intensity-based algorithm), the phase information from the image data (phase-based algorithm), or the complex image data (complex-based algorithm). An enface image is a 2D projection of 3D OCT data (e.g., by averaging the intensity of each individual A-scan, such that each A-scan defines a pixel in the 2D projection). Similarly, an en face vasculature image is an image displaying motion contrast signal in which the data dimension corresponding to depth (e.g., z-direction along an A-scan) is displayed as a single representative value (e.g., a pixel in a 2D projection image), typically by summing or integrating all or an isolated portion of the data (see for example U.S. Pat. No. 7,301,644 herein incorporated in its entirety by reference). OCT systems that provide an angiography imaging functionality may be termed OCT angiography (OCTA) systems.

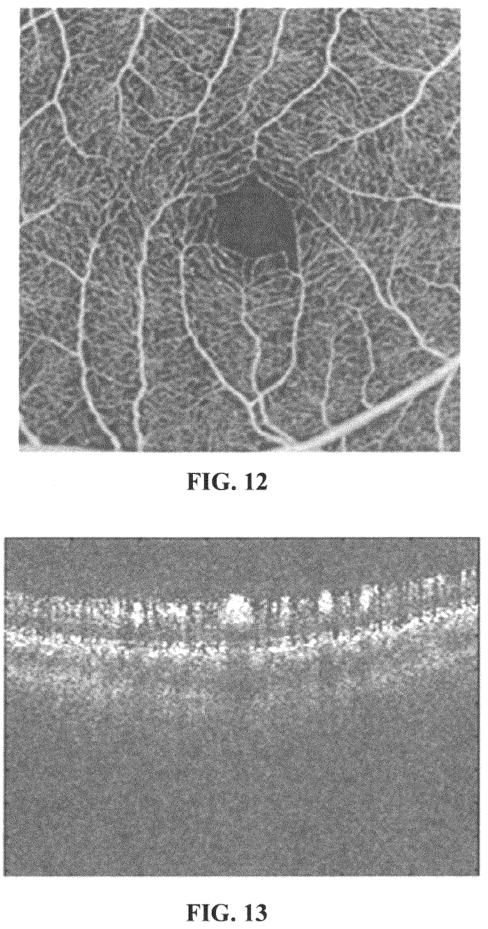

FIG. 12 shows an example of an enface vasculature image. After processing the data to highlight motion contrast using any of the motion contrast techniques known in the art, a range of pixels corresponding to a given tissue depth from the surface of internal limiting membrane (ILM) in retina, may be summed to generate the en face (e.g., frontal view) image of the vasculature. FIG. 13 shows an exemplary B-scan of a vasculature (OCTA) image. As illustrated, structural information may not be well-defined since blood flow may traverse multiple retinal layers making them less defined than in a structural OCT B-scan, as shown in FIG. 11. Nonetheless, OCTA provides a non-invasive technique for imaging the microvasculature of the retina and the choroid, which may be critical to diagnosing and/or monitoring various pathologies. For example, OCTA may be used to identify diabetic retinopathy by identifying microaneurysms, neovascular complexes, and quantifying foveal avascular zone and nonperfused areas. Moreover, OCTA has been shown to be in good agreement with fluorescein angiography (FA), a more traditional, but more evasive, technique requiring the injection of a dye to observe vascular flow in the retina. Additionally, in dry age-related macular degeneration, OCTA has been used to monitor a general decrease in choriocapillaris flow. Similarly in wet age-related macular degeneration, OCTA can provides a qualitative and quantitative analysis of choroidal neovascular membranes. OCTA has also been used to study vascular occlusions, e.g., evaluation of nonperfused areas and the integrity of superficial and deep plexus.

### Neural Networks

As discussed above, the present invention may use a neural network (NN) machine learning (ML) model. For the sake of completeness, a general discussion of neural networks is provided herein. The present invention may use any, singularly or in combination, of the below described neural network architecture(s). A neural network, or neural net, is a (nodal) network of interconnected neurons, where each neuron represents a node in the network. Groups of neurons may be arranged in layers, with the outputs of one layer feeding forward to a next layer in a multilayer perceptron (MLP) arrangement. MLP may be understood to be a feedforward neural network model that maps a set of input data onto a set of output data.

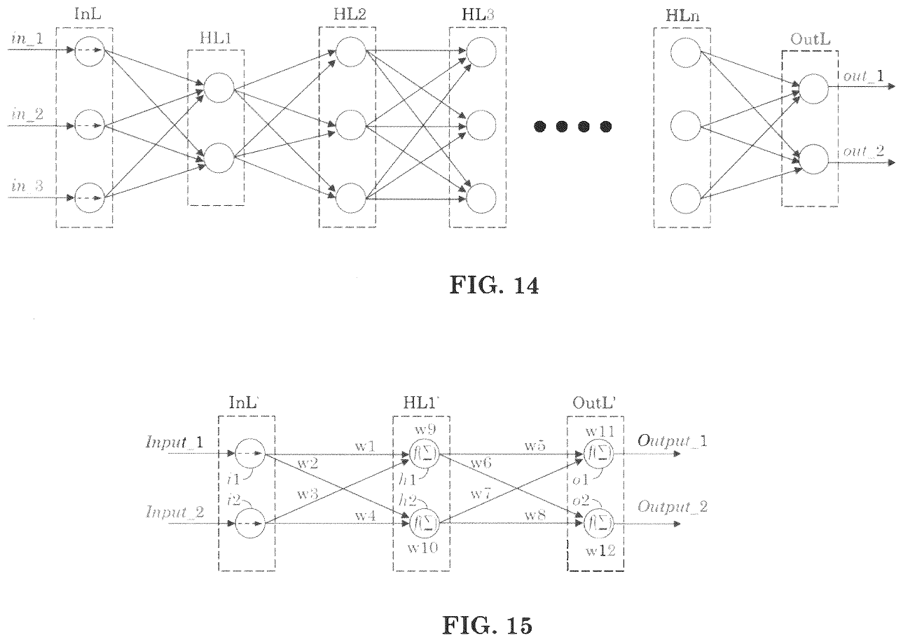

FIG. 14 illustrates an example of a multilayer perceptron (MLP) neural network. Its structure may include multiple hidden (e.g., internal) layers HL1 to HLn that map an input layer InL (that receives a set of inputs (or vector input) in_1 to in_3) to an output layer OutL that produces a set of outputs (or vector output), e.g., out_1 and out_2. Each layer may have any given number of nodes, which are herein illustratively shown as circles within each layer. In the present example, the first hidden layer HL1 has two nodes, while hidden layers HL2, HL3, and HLn each have three nodes. Generally, the deeper the MLP (e.g., the greater the number of hidden layers in the MLP), the greater its capacity to learn. The input layer InL receives a vector input (illustratively shown as a three-dimensional vector consisting of in_1, in_2 and in_3), and may apply the received vector input to the first hidden layer HL1 in the sequence of hidden layers. An output layer OutL receives the output from the last hidden layer, e.g., HLn, in the multilayer model, processes its inputs, and produces a vector output result (illustratively shown as a two-dimensional vector consisting of out_1 and out_2).

Typically, each neuron (or node) produces a single output that is fed forward to neurons in the layer immediately following it. But each neuron in a hidden layer may receive multiple inputs, either from the input layer or from the outputs of neurons in an immediately preceding hidden layer. In general, each node may apply a function to its inputs to produce an output for that node. Nodes in hidden layers (e.g., learning layers) may apply the same function to their respective input(s) to produce their respective output(s). Some nodes, however, such as the nodes in the input layer InL receive only one input and may be passive, meaning that they simply relay the values of their single input to their output(s), e.g., they provide a copy of their input to their output(s), as illustratively shown by dotted arrows within the nodes of input layer InL.

For illustration purposes, FIG. 15 shows a simplified neural network consisting of an input layer InL′, a hidden layer HL1′, and an output layer OutL′. Input layer InL′ is shown having two input nodes i1 and i2 that respectively receive inputs Input_1 and Input_2 (e.g. the input nodes of layer InL′ receive an input vector of two dimensions). The input layer InL′ feeds forward to one hidden layer HL1′ having two nodes h1 and h2, which in turn feeds forward to an output layer OutL′ of two nodes o1 and o2. Interconnections, or links, between neurons (illustrative shown as solid arrows) have weights w1 to w8. Typically, except for the input layer, a node (neuron) may receive as input the outputs of nodes in its immediately preceding layer. Each node may calculate its output by multiplying each of its inputs by each input's corresponding interconnection weight, summing the products of it inputs, adding (or multiplying by) a constant defined by another weight or bias that may be associated with that particular node (e.g., node weights w9, w10, w11, w12 respectively corresponding to nodes h1, h2, o1, and o2), and then applying a non-linear function or logarithmic function to the result. The non-linear function may be termed an activation function or transfer function. Multiple activation functions are known the art, and selection of a specific activation function is not critical to the present discussion. It is noted, however, that operation of the ML model, or behavior of the neural net, is dependent upon weight values, which may be learned so that the neural network provides a desired output for a given input.

The neural net learns (e.g., is trained to determine) appropriate weight values to achieve a desired output for a given input during a training, or learning, stage. Before the neural net is trained, each weight may be individually assigned an initial (e.g., random and optionally non-zero) value, e.g. a random-number seed. Various methods of assigning initial weights are known in the art. The weights are then trained (optimized) so that for a given training vector input, the neural network produces an output close to a desired (predetermined) training vector output. For example, the weights may be incrementally adjusted in thousands of iterative cycles by a technique termed back-propagation. In each cycle of back-propagation, a training input (e.g., vector input or training input image/sample) is fed forward through the neural network to determine its actual output (e.g., vector output). An error for each output neuron, or output node, is then calculated based on the actual neuron output and a target training output for that neuron (e.g., a training output image/sample corresponding to the present training input image/sample). One then propagates back through the neural network (in a direction from the output layer back to the input layer) updating the weights based on how much effect each weight has on the overall error so that the output of the neural network moves closer to the desired training output. This cycle is then repeated until the actual output of the neural network is within an acceptable error range of the desired training output for the given training input. As it would be understood, each training input may require many back-propagation iterations before achieving a desired error range. Typically, an epoch refers to one back-propagation iteration (e.g., one forward pass and one backward pass) of all the training samples, such that training a neural network may require many epochs. Generally, the larger the training set, the better the performance of the trained ML model, so various data augmentation methods may be used to increase the size of the training set. For example, when the training set includes pairs of corresponding training input images and training output images, the training images may be divided into multiple corresponding image segments (or patches). Corresponding patches from a training input image and training output image may be paired to define multiple training patch pairs from one input/output image pair, which enlarges the training set. Training on large training sets, however, places high demands on computing resources, e.g. memory and data processing resources. Computing demands may be reduced by dividing a large training set into multiple mini-batches, where the mini-batch size defines the number of training samples in one forward/backward pass. In this case, and one epoch may include multiple mini-batches. Another issue is the possibility of a NN overfitting a training set such that its capacity to generalize from a specific input to a different input is reduced. Issues of overfitting may be mitigated by creating an ensemble of neural networks or by randomly dropping out nodes within a neural network during training, which effectively removes the dropped nodes from the neural network. Various dropout regulation methods, such as inverse dropout, are known in the art.

It is noted that the operation of a trained NN machine model is not a straight-forward algorithm of operational/analyzing steps. Indeed, when a trained NN machine model receives an input, the input is not analyzed in the traditional sense. Rather, irrespective of the subject or nature of the input (e.g., a vector defining a live image/scan or a vector defining some other entity, such as a demographic description or a record of activity) the input will be subjected to the same predefined architectural construct of the trained neural network (e.g., the same nodal/layer arrangement, trained weight and bias values, predefined convolution/deconvolution operations, activation functions, pooling operations, etc.), and it may not be clear how the trained network's architectural construct produces its output. Furthermore, the values of the trained weights and biases are not deterministic and depend upon many factors, such as the amount of time the neural network is given for training (e.g., the number of epochs in training), the random starting values of the weights before training starts, the computer architecture of the machine on which the NN is trained, selection of training samples, distribution of the training samples among multiple mini-batches, choice of activation function(s), choice of error function(s) that modify the weights, and even if training is interrupted on one machine (e.g., having a first computer architecture) and completed on another machine (e.g., having a different computer architecture). The point is that the reasons why a trained ML, model reaches certain outputs is not clear, and much research is currently ongoing to attempt to determine the factors on which a ML model bases its outputs. Therefore, the processing of a neural network on live data cannot be reduced to a simple algorithm of steps. Rather, its operation is dependent upon its training architecture, training sample sets, training sequence, and various circumstances in the training of the MIL model.

In summary, construction of a NN machine learning model may include a learning (or training) stage and a classification (or operational) stage. In the learning stage, the neural network may be trained for a specific purpose and may be provided with a set of training examples, including training (sample) inputs and training (sample) outputs, and optionally including a set of validation examples to test the progress of the training. During this learning process, various weights associated with nodes and node-interconnections in the neural network are incrementally adjusted in order to reduce an error between an actual output of the neural network and the desired training output. In this manner, a multilayer feed-forward neural network (such as discussed above) may be made capable of approximating any measurable function to any desired degree of accuracy. The result of the learning stage is a (neural network) machine learning (IL) model that has been learned (e.g., trained). In the operational stage, a set of test inputs (or live inputs) may be submitted to the learned (trained) MIL model, which may apply what it has learned to produce an output prediction based on the test inputs.

Like the regular neural networks of FIGS. 14 and 15, convolutional neural networks (CNN) are also made up of neurons that have learnable weights and biases. Each neuron receives inputs, performs an operation (e.g., dot product), and is optionally followed by a non-linearity. The CNN, however, may receive raw image pixels at one end (e.g., the input end) and provide classification (or class) scores at the other end (e.g., the output end). Because CNNs expect an image as input, they are optimized for working with volumes (e.g., pixel height and width of an image, plus the depth of the image, e.g., color depth such as an RGB depth defined of three colors: red, green, and blue). For example, the layers of a CNN may be optimized for neurons arranged in 3 dimensions. The neurons in a CNN layer may also be connected to a small region of the layer before it, instead of all of the neurons in a fully-connected NN. The final output layer of a CNN may reduce a full image into a single vector (classification) arranged along the depth dimension.

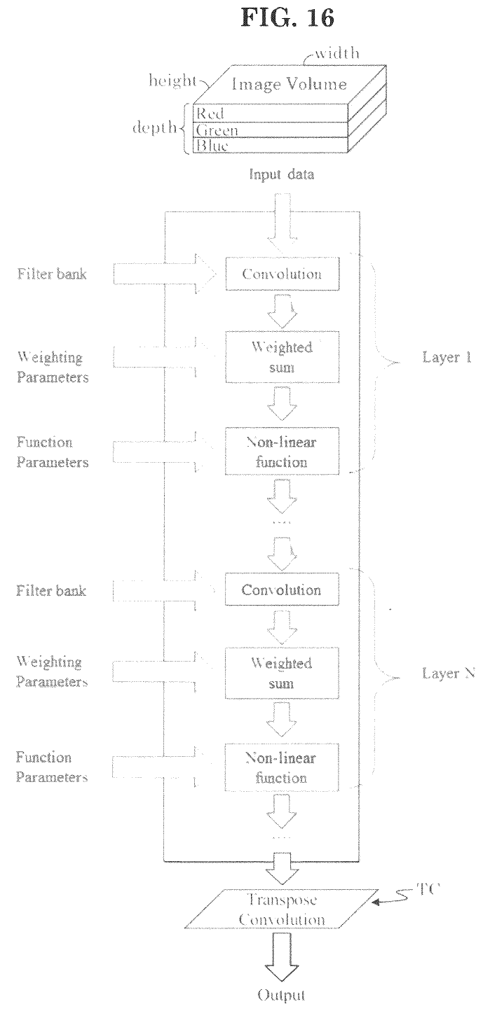

FIG. 16 provides an example convolutional neural network architecture. A convolutional neural network may be defined as a sequence of two or more layers (e.g., Layer 1 to Layer N), where a layer may include a (image) convolution step, a weighted sum (of results) step, and a non-linear function step. The convolution may be performed on its input data by applying a filter (or kernel), e.g. on a moving window across the input data, to produce a feature map. Each layer and component of a layer may have different predetermined filters (from a filter bank), weights (or weighting parameters), and/or function parameters. In the present example, the input data is an image, which may be raw pixel values of the image, of a given pixel height and width. In the present example, the input image is illustrated as having a depth of three color channels RGB (Red, Green, and Blue). Optionally, the input image may undergo various preprocessing, and the preprocessing results may be input in place of, or in addition to, the raw input image. Some examples of image preprocessing may include: retina blood vessel map segmentation, color space conversion, adaptive histogram equalization, connected components generation, etc. Within a layer, a dot product may be computed between the given weights and a small region they are connected to in the input volume. Many ways of configuring a CNN are known in the art, but as an example, a layer may be configured to apply an elementwise activation function, such as max (0,x) thresholding at zero. A pooling function may be performed (e.g., along the x-y directions) to down-sample a volume. A fully-connected layer may be used to determine the classification output and produce a one-dimensional output vector, which has been found useful for image recognition and classification. However, for image segmentation, the CNN would need to classify each pixel. Since each CNN layers tends to reduce the resolution of the input image, another stage is needed to up-sample the image back to its original resolution. This may be achieved by application of a transpose convolution (or deconvolution) stage TC, which typically does not use any predefine interpolation method, and instead has learnable parameters.

Convolutional Neural Networks have been successfully applied to many computer vision problems. As explained above, training a CNN generally requires a large training dataset. The U-Net architecture is based on CNNs and can generally be trained on a smaller training dataset than conventional CNNs.

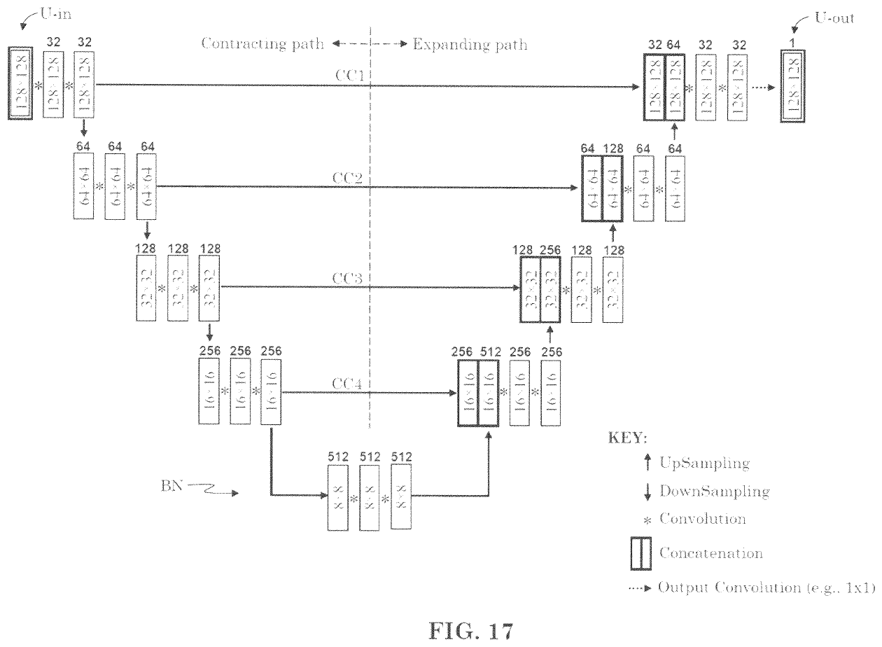

FIG. 17 illustrates an example U-Net architecture. The present exemplary U-Net includes an input module (or input layer or stage) that receives an input U-in (e.g., input image or image patch) of any given size. For illustration purposes, the image size at any stage, or layer, is indicated within a box that represents the image, e.g., the input module encloses number “128×128” to indicate that input image U-in is comprised of 128 by 128 pixels. The input image may be a fundus image, an OCT/OCTA enface, B-scan image, etc. It is to be understood, however, that the input may be of any size or dimension. For example, the input image may be an RGB color image, monochrome image, volume image, etc. The input image undergoes a series of processing layers, each of which is illustrated with exemplary sizes, but these sizes are illustration purposes only and would depend, for example, upon the size of the image, convolution filter, and/or pooling stages. The present architecture consists of a contracting path (herein illustratively comprised of four encoding modules) followed by an expanding path (herein illustratively comprised of four decoding modules), and copy-and-crop links (e.g., CC1 to CC4) between corresponding modules/stages that copy the output of one encoding module in the contracting path and concatenates it to (e.g., appends it to the back of) the up-converted input of a correspond decoding module in the expanding path. This results in a characteristic U-shape, from which the architecture draws its name. Optionally, such as for computational considerations, a “bottleneck” module/stage (BN) may be positioned between the contracting path and the expanding path. The bottleneck BN may consist of two convolutional layers (with batch normalization and optional dropout).

The contracting path is similar to an encoder, and generally captures context (or feature) information by the use of feature maps. In the present example, each encoding module in the contracting path may include two or more convolutional layers, illustratively indicated by an asterisk symbol “*”, and which may be followed by a max pooling layer (e.g., DownSampling layer). For example, input image U-in is illustratively shown to undergo two convolution layers, each with 32 feature maps. As it would be understood, each convolution kernel produces a feature map (e.g., the output from a convolution operation with a given kernel is an image typically termed a “feature map”). For example, input U-in undergoes a first convolution that applies 32 convolution kernels (not shown) to produce an output consisting of 32 respective feature maps. However, as it is known in the art, the number of feature maps produced by a convolution operation may be adjusted (up or down). For example, the number of feature maps may be reduced by averaging groups of feature maps, dropping some feature maps, or other known method of feature map reduction. In the present example, this first convolution is followed by a second convolution whose output is limited to 32 feature maps. Another way to envision feature maps may be to think of the output of a convolution layer as a 3D image whose 2D dimension is given by the listed X-Y planar pixel dimension (e.g., 128×128 pixels), and whose depth is given by the number of feature maps (e.g., 32 planar images deep). Following this analogy, the output of the second convolution (e.g., the output of the first encoding module in the contracting path) may be described as a 128×128×32 image. The output from the second convolution then undergoes a pooling operation, which reduces the 2D dimension of each feature map (e.g., the X and Y dimensions may each be reduced by half). The pooling operation may be embodied within the DownSampling operation, as indicated by a downward arrow. Several pooling methods, such as max pooling, are known in the art and the specific pooling method is not critical to the present invention. The number of feature maps may double at each pooling, starting with 32 feature maps in the first encoding module (or block), 64 in the second encoding module, and so on. The contracting path thus forms a convolutional network consisting of multiple encoding modules (or stages or blocks). As is typical of convolutional networks, each encoding module may provide at least one convolution stage followed by an activation function (e.g., a rectified linear unit (ReLU) or sigmoid layer), not shown, and a max pooling operation. Generally, an activation function introduces non-linearity into a layer (e.g., to help avoid overfitting issues), receives the results of a layer, and determines whether to “activate” the output (e.g., determines whether the value of a given node meets predefined criteria to have an output forwarded to a next layer/node). In summary, the contracting path generally reduces spatial information while increasing feature information.

The expanding path is similar to a decoder, and among other things, may provide localization and spatial information for the results of the contracting path, despite the down sampling and any max-pooling performed in the contracting stage. The expanding path includes multiple decoding modules, where each decoding module concatenates its current up-converted input with the output of a corresponding encoding module. In this manner, feature and spatial information are combined in the expanding path through a sequence of up-convolutions (e.g., UpSampling or transpose convolutions or deconvolutions) and concatenations with high-resolution features from the contracting path (e.g., via CC1 to CC4). Thus, the output of a deconvolution layer is concatenated with the corresponding (optionally cropped) feature map from the contracting path, followed by two convolutional layers and activation function (with optional batch normalization).

The output from the last expanding module in the expanding path may be fed to another processing/training block or layer, such as a classifier block, that may be trained along with the U-Net architecture. Alternatively, or in addition, the output of the last upsampling block (at the end of the expanding path) may be submitted to another convolution (e.g., an output convolution) operation, as indicated by a dotted arrow, before producing its output U-out. The kernel size of output convolution may be selected to reduce the dimensions of the last upsampling block to a desired size. For example, the neural network may have multiple features per pixels right before reaching the output convolution, which may provide a 1×1 convolution operation to combine these multiple features into a single output value per pixel, on a pixel-by-pixel level.

### Computing Device/System

FIG. 18 illustrates an example computer system (or computing device or computer device). In some embodiments, one or more computer systems may provide the functionality described or illustrated herein and/or perform one or more steps of one or more methods described or illustrated herein. The computer system may take any suitable physical form. For example, the computer system may be an embedded computer system, a system-on-chip (SOC), a single-board computer system (SBC) (such as, for example, a computer-on-module (COM) or system-on-module (SOM)), a desktop computer system, a laptop or notebook computer system, a mesh of computer systems, a mobile telephone, a personal digital assistant (PDA), a server, a tablet computer system, an augmented/virtual reality device, or a combination of two or more of these. Where appropriate, the computer system may reside in a cloud, which may include one or more cloud components in one or more networks.

In some embodiments, the computer system may include a processor Cpnt1, memory Cpnt2, storage Cpnt3, an input/output (I/O) interface Cpnt4, a communication interface Cpnt5, and a bus Cpnt6. The computer system may optionally also include a display Cpnt7, such as a computer monitor or screen.

Processor Cpnt1 includes hardware for executing instructions, such as those making up a computer program. For example, processor Cpnt1 may be a central processing unit (CPU) or a general-purpose computing on graphics processing unit (GPGPU). Processor Cpnt1 may retrieve (or fetch) the instructions from an internal register, an internal cache, memory Cpnt2, or storage Cpnt3, decode and execute the instructions, and write one or more results to an internal register, an internal cache, memory Cpnt2, or storage Cpnt3. In particular embodiments, processor Cpnt1 may include one or more internal caches for data, instructions, or addresses. Processor Cpnt1 may include one or more instruction caches, one or more data caches, such as to hold data tables. Instructions in the instruction caches may be copies of instructions in memory Cpnt2 or storage Cpnt3, and the instruction caches may speed up retrieval of those instructions by processor Cpnt1. Processor Cpnt1 may include any suitable number of internal registers, and may include one or more arithmetic logic units (ALUs). Processor Cpnt1 may be a multi-core processor; or include one or more processors Cpnt1. Although this disclosure describes and illustrates a particular processor, this disclosure contemplates any suitable processor.

Memory Cpnt2 may include main memory for storing instructions for processor Cpnt1 to execute or to hold interim data during processing. For example, the computer system may load instructions or data (e.g., data tables) from storage Cpnt3 or from another source (such as another computer system) to memory Cpnt2. Processor Cpnt1 may load the instructions and data from memory Cpnt2 to one or more internal register or internal cache. To execute the instructions, processor Cpnt1 may retrieve and decode the instructions from the internal register or internal cache. During or after execution of the instructions, processor Cpnt1 may write one or more results (which may be intermediate or final results) to the internal register, internal cache, memory Cpnt2 or storage Cpnt3. Bus Cpnt6 may include one or more memory buses (which may each include an address bus and a data bus) and may couple processor Cpnt1 to memory Cpnt2 and/or storage Cpnt3. Optionally, one or more memory management unit (MMU) facilitate data transfers between processor Cpnt1 and memory Cpnt2. Memory Cpnt2 (which may be fast, volatile memory) may include random access memory (RAM), such as dynamic RAM (DRAM) or static RAM (SRAM). Storage Cpnt3 may include long-term or mass storage for data or instructions. Storage Cpnt3 may be internal or external to the computer system, and include one or more of a disk drive (e.g., hard-disk drive, HDD, or solid-state drive, SSD), flash memory, ROM, EPROM, optical disc, magneto-optical disc, magnetic tape, Universal Serial Bus (USB)-accessible drive, or other type of non-volatile memory.

I/O interface Cpnt4 may be software, hardware, or a combination of both, and include one or more interfaces (e.g., serial or parallel communication ports) for communication with I/O devices, which may enable communication with a person (e.g., user). For example, I/O devices may include a keyboard, keypad, microphone, monitor, mouse, printer, scanner, speaker, still camera, stylus, tablet, touch screen, trackball, video camera, another suitable I/O device, or a combination of two or more of these.

Communication interface Cpnt5 may provide network interfaces for communication with other systems or networks. Communication interface Cpnt5 may include a Bluetooth interface or other type of packet-based communication. For example, communication interface Cpnt5 may include a network interface controller (NIC) and/or a wireless NIC or a wireless adapter for communicating with a wireless network. Communication interface Cpnt5 may provide communication with a WI-FI network, an ad hoc network, a personal area network (PAN), a wireless PAN (e.g., a Bluetooth WPAN), a local area network (LAN), a wide area network (WAN), a metropolitan area network (MAN), a cellular telephone network (such as, for example, a Global System for Mobile Communications (GSM) network), the Internet, or a combination of two or more of these.

Bus Cpnt6 may provide a communication link between the above-mentioned components of the computing system. For example, bus Cpnt6 may include an Accelerated Graphics Port (AGP) or other graphics bus, an Enhanced Industry Standard Architecture (EISA) bus, a front-side bus (FSB), a HyperTransport (HT) interconnect, an Industry Standard Architecture (ISA) bus, an InfiniBand bus, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or other suitable bus or a combination of two or more of these.

Although this disclosure describes and illustrates a particular computer system having a particular number of particular components in a particular arrangement, this disclosure contemplates any suitable computer system having any suitable number of any suitable components in any suitable arrangement.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

While the invention has been described in conjunction with several specific embodiments, it is evident to those skilled in the art that many further alternatives, modifications, and variations will be apparent in light of the foregoing description. Thus, the invention described herein is intended to embrace all such alternatives, modifications, applications and variations as may fall within the spirit and scope of the appended claims.

1. A method for implementing a transaction of medical data, comprising:
with agreement of an owner of the medical data, a managing entity receiving the medical data for storage and recording a summary of the received medical data to a blockchain computer network maintaining an electronic ledger of available medical records;
the managing entity responding to an electronic request from a remote user for medical data meeting user-specified criteria, by providing the remote user with a listing of available qualifying medical records that meet the user-specified criteria, as determined at least in part from the electronic ledger;
the managing entity responding to the remote user selecting one or more of the qualifying medical records by granting the remote user access to the selected qualifying medical records in accordance with an access-approval status for each qualifying medical record granted by the owner of the qualifying medical record and recording the data transaction to the blockchain computer network.

2. The method of claim 1, wherein:
the managing entity further receives from the remote user an access-type request indicating a type of data access being requested;
the access-type request including one or more of data viewing, temporary data access with expiration period, permanent data access with data download, and data processing remote from the remote user and managed by the managing entity.

3. The method of claim 2, wherein permanent access with data download includes removal of the accessed qualifying medical data from the listing of available medical records in the electronic ledger.

4. The method of claim 2, wherein each access-type request has an associated access-price payable by the remote user.

5. The method of claim 2, wherein data processing managed by the managing entity includes generation of a machine learning model using the selected medical records and granting the remote user access to the generated machine learning model.

6. The method of claim 5, wherein the generated machine learning model includes a deep learning model selected from one or more of an artificial neural network, convolutional neural network, u-net, recurrent neural networks, generative adversarial networks, and multilayer perceptrons.

7. The method of claim 5, wherein the generated machine learning model includes a machine learning model based one or more of a classification model, regression model, clustering, and dimensionality reduction.

8. The method of claim 1, wherein the owner of the medical data is the patient described by the medical data.

9. The method of claim 1, wherein the medical data includes medical measurements or images taken by a medical device, and the medical device automatically sends the medical data to the managing entity with consent from the patient.

10. The method of claim 1 wherein the owner of the medical data is identified by a public identifier based on a public key that is associated with a corresponding private key, the public identifier excluding any personal identifying information, whereby the managing entity maintains hidden any personal identifying information of the owner of the medical data.

11. The method of claim 1, wherein:
the listing of available qualifying medical records that meet the user-specified criteria includes a count of qualifying medical records; and
the managing entity responds to an electronic altering of the user-specified criteria by providing the remote user with an updated listing of available qualifying medical records that meet the altered user-specified criteria.

12. The method of claim 1, wherein the managing entity:
in response to the remote user selecting one or more of the qualifying medical records, checking for a pre-existing access-approval status for each of the selected qualifying medical records; and
for each selected qualifying medical record not having a pre-existing access-approval status, transmitting a request for approval to the owner of the qualifying medical record, and updating the approval status of the qualifying medical record in accordance with the owner's approval response.

13. The method of claim 1, wherein the managing entity:
in response to the remote user specifying a desired number of qualifying medical records, checking for a pre-existing access-approval status for each of the qualifying medical records; and
in response to a sufficient number of qualifying medical records having a pre-existing access-approval status, selecting the desired number of qualifying medical records from among those having a pre-existing access-approval status;
in response to an insufficient number of qualifying medical records having a pre-existing access-approval status, determining how many additional qualifying medical records are needed to meet the specified desired number, and for each additionally needed qualifying medical record, transmitting a request for approval to the owner of the additionally needed qualifying medical record, and updating the approval status of the additionally needed qualifying medical record in accordance with the owner's approval response.

14. The method of claim 1, wherein the managing entity:
responds to the electronic request from the remote user for medical data meeting user-specified criteria, by providing the remote user with a price list associate with the listing of available qualifying medical records; and
responds to the remote user selecting one or more of the qualifying medical records, by collecting the price associated with the qualifying medical records to which the remote user is granted access, and distributing a payment to one or more of each selected qualified medical record owner, medical institution that collected any of the qualifying medical records to which the remote user was granted access, and data host that hosts any of the qualifying medical records to which the remote user was granted access.

15. The method of claim 1, wherein the blockchain computer network is a public ledger computer network.

16. The method of claim 1, wherein the received medical data is at least partly stored on-chain within the blockchain computer network and off-chain external to the blockchain computer network.

17. The method of claim 1, wherein in response to the remote user being a doctor to which a patient is being referred to, and the user-specified criteria specifies medical records of the patient only, the managing entity maintains an automatic access-approval status for the qualifying medical records.

18. The method of claim 1, wherein in response to the remote user being the owner of the medical data and the user-specified criteria specifying only medical records of the owner of the medical data, the managing entity maintains an automatic access-approval status for the qualifying medical record.

19. The method of claim 1, wherein the managing entity maintains a group of privileged remote users, each of which has unimpeded review-access to the electronic ledger.

20. The method of claim 19, wherein the privileged users are identified based on access-criteria established by the managing entity, including one or more of prior given-authorization, number of medical records submitted by the privileged users for storage to the managing entity, and prior agreed-upon subscription period.

21. The method of claim 1, wherein the user-specified criteria submitted by the remote user includes one or more of a type of anatomical measurement, body function measurement, a data scan type, and an image type.

22. The method of claim 21, wherein:
the anatomical measurement includes one or more of eye pressure, keratometry measurements, refractive error, or eye size;
the body function measurement includes one or more of an electrocardiogram, body temperature, pulse rate, or respiration rate;
the data scan type includes one or more of an A-scan, B-scan, or C-scan from an optical coherence tomography device; and
the image type includes one or more of a fundus image, an en-face image, or an ophthalmic anterior segment image.
